(19) United States
(12) Patent Application Publication
(10) Pub. No.: US 2021/0346506 A1
(43) Pub. Date: Nov. 11, 2021

(54) PRODRUG OF ITRACONAZOLE AND USES THEREOF

(71) Applicant: Guibai LIANG, Scotch Plains, NJ (US)

(72) Inventor: Guibai LIANG, Scotch Plains, NJ (US)

(21) Appl. No.: 17/308,160

(22) Filed: May 5, 2021

Related U.S. Application Data
(60) Provisional application No. 63/021,201, filed on May 7, 2020.

(51) Int. Cl. A61K 47/54; A61K 31/496; A61K 9/00

(52) U.S. Cl. CPC A61K 47/548; A61K 9/0053; A61K 9/0019; A61K 31/496

(57) ABSTRACT

The present invention relates to itraconazole prodrugs, pharmaceutical compositions comprising the prodrugs, and methods for treatment and/or prophylaxis of lung fibrosis, renal fibrosis, or liver fibrosis using the pharmaceutical compositions.

### RELATED APPLICATIONS AND INCORPORATION BY REFERENCE

This application claims priority to U.S. Provisional Application No. 63/021,201 filed May 7, 2020.

All documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### FIELD OF THE INVENTION

The present disclosure relates to itraconazole prodrugs and pharmaceutical compositions comprising the same. The present disclosure further provides a method for treatment and/or prophylaxis of lung fibrosis, renal fibrosis, or liver fibrosis using the pharmaceutical compositions.

### BACKGROUND OF THE INVENTION

Lung fibrosis, renal fibrosis, and liver fibrosis are serious illnesses with heavy healthcare burdens. Among them, Idiopathic Pulmonary Fibrosis (IPF) is the most common cause of death from progressive lung disease. It affects about 5 million people worldwide. An estimated median survival after diagnosis is only 2-3 years (Chakraborty et al., (2014) Expert Opin Investig Drugs, 23:893-910; Spagnolo et al., (2015) Pharmacology & Therapeutics 152:18-27; Tzouvelekis et al., (2015) Therapeutics and Clinical Risk Management 11:359-370).

The etiology of IPF remains unknown. Potential factors, such as cigarette smoking, dust exposure and infection agents, however, have been associated with the development of IPF. IPF is characterized by progressive and irreversible distortion of the lung's architecture as a result of apoptosis of epithelial and endothelial cells, fibroblast hyperplasia and extracellular metric remodeling (Chakraborty et al., (2014) supra).

Development of agents for treatment of IPF has been slow in progress. The first two agents for treating IPF, pirfenidone and nintedanib were approved only at the end of 2014 (King et al., (2014) N Engl J Med 370:2083-92; Richeldi et al., (2014) N Engl J Med 370:2071-82). These two agents, however, have only significant side effects and require complicated dosing regimen. Therefore, there remains a significant need for IPF treatment methods and compositions.

Itraconazole, a FDA approved imidazole/triazole type antifungal agent, has been found to be highly efficacious against IPF at a relatively low dose with comparable anti-fibrotic activity to nintedanib and no observed adverse effect (US2019282565A1). To keep itraconazole at a low level within the body is important, as a high concentration may bring adverse effects to human health. As is known in the art, itraconazole is a highly selective inhibitor of cytochrome P-450 sterol C-14 α-demethylation (Perfect J R, (2017) Nature Review Drug Discovery 16:603-616), and has inhibitory activity toward both the hedgehog signaling pathway (Kim J et al., (2010) Cancer Cell. 17:388-399; Horn A et al., (2012) Arthritis Rheum. 64:2724-2733; Bolanos A L et al., (2012) Am J Physiol Lung Cell Mol Physiol. 303:L978-L990) and angiogenesis (Chong et al., (2007) ACS Chem Biol. 2:263-70).

Citation or identification of any document in this application is not an admission that such document is available as prior art to the present invention.

### SUMMARY OF THE INVENTION

The present invention relates to itraconazole prodrug compounds, which are stable in mouse and human blood and slowly converted to itraconazole. Administration of such prodrugs enable sustained release of the active itraconazole compound at an efficacious but safe level.

In a first aspect, the present disclosure provides an itraconazole prodrug compound represented by Formula (I), or a pharmaceutically acceptable salt or solvent thereof,

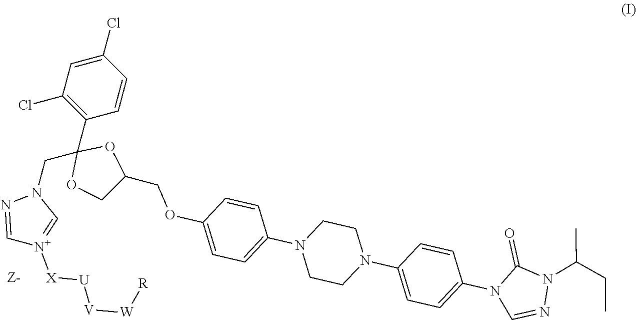

wherein X is omitted, or represents —CH₂— optionally substituted by C₁-C₅ alkyl,
U is omitted, or represents —O—, —S—, —NH—, or —N(R¹)—,
V is omitted, or represents —CO—, —SO—, —SO₂—, or

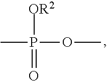

W is omitted, or represents —O—, —S—, —NH—, or —N(R³)—,
R represents H, C₁-C₅ alkyl, or hydroxyl-C₁-C₅ alkyl,
R¹, R² and R³ independently represents C₁-C₅ alkyl, or hydroxyl-C₁-C₅ alkyl,
Z⁻ represents F⁻, Cl⁻, Br⁻, I⁻, or another pharmaceutically acceptable anion, including, but not limited to, PO₃⁻, SO₄⁻, HCO₂⁻, CH₃COO⁻, and CH₄H₂O₄²⁻.

In one embodiment, X, U, V, and W are omitted, R is alkyl or hydroxyalkyl, and Z⁻ represents F⁻, Cl⁻, Br⁻, I⁻, or another pharmaceutically acceptable anion. In one embodiment, X, U, V, and W are omitted, R is —CH₃, and Z⁻ is Cl⁻. In one embodiment, X, U, V, and W are omitted, R is —CH₂CH₃, and Z⁻ is Cl⁻.

In another embodiment, X is —CH₂—, U is —O—, V is —CO—, W is omitted, R is alkyl or hydroxyalkyl, and Z⁻ represents F⁻, Cl⁻, Br⁻, I⁻, or another pharmaceutically acceptable anion. In one embodiment, X is —CH₂—, U is —O—, V is —CO—, W is omitted, R is —CH₃, and Z⁻ is Cl⁻. In one embodiment, X is —CH₂—, U is —O—, V is —CO—, W is omitted, R is —CH(CH₃)CH₃, and Z⁻ is Cl⁻. In one embodiment, X is —CH₂—, U is —O—, V is —CO—, W is omitted, R is —C(CH₃)₃, and Z⁻ is Cl⁻.

In another embodiment, X is —CH₂—, U is —O—, V is —CO—, W is —O—, R is alkyl or hydroxyalkyl, and Z⁻ represents F⁻, Cl⁻, Br⁻, I⁻, or another pharmaceutically acceptable anion. In one embodiment, X is —CH₂—, U is —O—, V is —CO—, W is —O—, R is —CH₂CH₃, and Z⁻ is Cl⁻. In one embodiment, X is —CH₂—, U is —O—, V is —CO—, W is —O—, R is —CH₂CH₂CH₃, and Z⁻ is Cl⁻. In one embodiment, X is —CH₂—, U is —O—, V is —CO—, W is —O—, R is —CH(CH₃)₂, and Z⁻ is Cl⁻. In one embodiment, X is —CH₂—, U is —O—, V is —CO—, W is —O—, R is —CH₂CH₂CH₂OH, and Z⁻ is Cl⁻.

In another embodiment, X is —CH₂—, U is —O—, V is

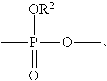

W is omitted, R and R² are independently alkyl or hydroxyalkyl, and Z⁻ represents F⁻, Cl⁻, Br⁻, I⁻, or another pharmaceutically acceptable anion. In one embodiment, X is —CH₂—, U is —O—, V is

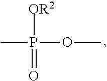

R² is —CH₂CH₃, W is omitted, R is —CH₂CH₃, and Z⁻ is Cl⁻.

The optically pure enantiomers, mixtures of enantiomers such as racemates, diastereomers, mixtures of diastereomer, diastereomeric racemates, mixtures of diastereomeric racemates, meso-forms, and morphological forms of the compound of Formula (I) are also encompassed in the disclosure.

In a second aspect, the present disclosure provides a pharmaceutical composition comprising a therapeutically effective amount of a compound of Formula (I), and a pharmaceutically acceptable carrier.

The pharmaceutical composition can be used for treatment and/or prophylaxis of lung fibrosis, renal fibrosis, liver fibrosis, and and complications resulting from such diseases. The composition may be in an oral or inhaler dosage, and the inhaler dosage, in some embodiments, is more effective than the oral dosage, with fewer side effects for all forms of lung fibrosis.

In a third aspect, the present disclosure provides a method for treatment and/or prophylaxis of lung fibrosis, renal fibrosis, liver fibrosis, and and complications resulting from such diseases, comprising administering a subject in need thereof the pharmaceutical composition of the disclosure.

In one embodiment, the method is for treatment and/or prophylaxis of IPF.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A and 1B show the stability of Compound 3 and itraconazole release in mouse (A) and human (B) blood.

### DETAILED DESCRIPTION OF THE INVENTION

To ensure that the present disclosure may be more readily understood, certain terms are first defined. Additional definitions are set forth throughout the detailed description.

The singular forms “a,” “an,” and “the” include plural referents unless the context clearly dictates otherwise.

“Itraconazole” is a common name for a triazole antifungal compound, the specific chemical structure and IUPAC name of which are well known in the art. It is available commercially (see Merck Index Reg. No. 5262 (12th ed. 1996) and U.S. Pat. No. 4,267,179). As used herein, “itraconazole” includes not only the chemical compound (free base form, also referred to as “free itraconazole”), but also all optical isomers, such as enantiomers, diastereomers, meso compounds, and the like, as well as pharmaceutically acceptable salts, solvates, and prodrugs thereof.

A “reference composition of itraconazole” (reference composition) is a composition comprising itraconazole that exhibits one or more of (1) has a AUCₜ in the fasted state that is about 35% or more lower than the AUCₜ in the fed state; (2) has an intra-subject variability of about 30% or greater; and (3) about 100 mg of itraconazole or more. Particular reference compositions include those with about 100 mg of itraconazole or more. Other particular reference compositions include those that do not include a solid solution or solid dispersion of itraconazole in an acid resistant polymeric carrier. One exemplary particular reference composition contains a blend of itraconazole, and one or more excipients, such as diluents, carriers, fillers, disintegrants, and the like. Another exemplary particular reference composition contains 100 mg of itraconazole, sugar spheres, hydroxypropyl methyl cellulose, and polyethylene glycol, such as polyethylene glycol 20000, in a gelatin capsule shell. For example, and most particularly, the reference dosage form can be an itraconazole capsule commercially available under the name SPORANOX®.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Cis- and trans- (or E- and Z-) geometric isomers of the compounds of the present disclosure are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization. Depending on the process conditions the end products of the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds of the present disclosure may be separated into the individual isomers. Compounds of the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure.

As referred to herein, the term “substituted” means that at least one hydrogen atom is replaced with a non-hydrogen group, provided that normal valencies are maintained and that the substitution results in a stable compound. When a substituent is noted as “optionally substituted”, the hydrogen atom may be or may be not replaced by a non-hydrogen group.

As used herein, the term “alkyl” or “alkylene” is intended to include both branched and straight-chain saturated aliphatic hydrocarbon groups having the specified number of carbon atoms. For example, “C₁-C₅ alkyl” denotes alkyl having 1 to 5 carbon atoms. Exemplary alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “hydroxy-alkyl”, alone or in combination with other groups, refers to an HO—R group, wherein R is alkyl. The term “hydroxy-C₁-C₅ alkyl” refers to an HO—R, wherein R is C₁-C₅ alkyl. Examples include, but not limited to, hydroxymethyl, hydroxyethyl and hydroxypropyl.

The phrase “pharmaceutically acceptable” is employed herein to refer to those compounds, and compositions that are, within the scope of sound medical judgment, suitable for use in contact with the tissues of human beings and animals without excessive toxicity, irritation, allergic response, and/or other problem or complication, commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. A “pharmaceutically acceptable solvate” refers to a multicomponent crystalline solid molecular adduct containing the host molecule (e.g., the compound of Formula (I)) and guest solvent molecule(s) incorporated in the crystal lattice structure. When the solvent is water, the solvate is called hydrate. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22nd Edition, Allen, L. V. Jr., Ed.; Pharmaceutical Press, London, UK (2012), the disclosure of which is hereby incorporated by reference.

The term “prodrug” refers to a biologically inactive compound that, after administration, is metabolized into a pharmacologically active drug inside human body. In the present disclosure, the prodrug is converted through metabolic process into itraconazole.

As used herein, the term “effective amount” means that amount of a drug or pharmaceutical agent, i.e., a prodrug compound of the disclosure, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. An effective amount can be administered in one or more administrations, applications or dosages and is not intended to be limited to a particular formulation or administration route. The term also includes within its scope amounts effective to enhance normal physiological function.

“Therapeutically effective amount” or “effective amount” refers the amount of a pharmaceutically active agent, such as itraconazole, that, when administered to a patient for treating a disease according to the dosing regimen as described herein, is sufficient to effect such treatment for the disease. The “therapeutically effective amount” will vary depending on the disease and its severity, and the age, weight, and other conditions of the patient to be treated.

A composition or dosage form is “therapeutically equivalent” to a reference composition or dosage form if it has a therapeutic effect that is substantially similar to the therapeutic effect of the reference composition or dosage form, for example, therapeutically equivalent dosage forms can have substantially similar efficacy towards a particular disease or condition when administered over a substantially similar time period. As used herein, the term “treating” or “treatment” includes any effect, e.g., lessening, reducing, modulating, ameliorating or eliminating, that results in the improvement of the condition, disease, disorder, and the like, or ameliorating a symptom thereof.

The term “prophylaxis” means to the prevention of a specific disease, by, e.g., studying the presence or level of the causative agent and applying a series of measures against it.

As used herein, the term “IPF” or “idiopathic pulmonary fibrosis” includes all forms of idiopathic pulmonary fibrosis, including, but not limited to, occupational and environmental, auto-immune, scleroderma, sarcoidosis, drug- and radiation-induced, genetic/familial fibrosis.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “half life” or “T_{1/2}” of a compound refers to the time it takes from its maximum concentration to half maximum concentration in human body.

Some exemplary prodrug compounds of Formula (I) are set forth below.

| Compound | Structure |
|---|---|
| Compound 1 | 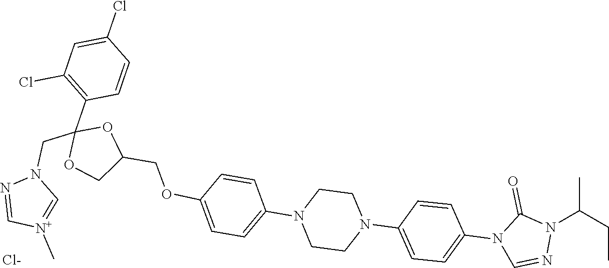 [SMILES: CCC(C)n1ncn(-c2ccc(N3CCN(c4ccc(OCC5COC(Cn6c[n+](C)cn6)(c6ccc(Cl)cc6Cl)O5)cc4)CC3)cc2)c1=O ; [Cl-]] |
| Compound 2 | 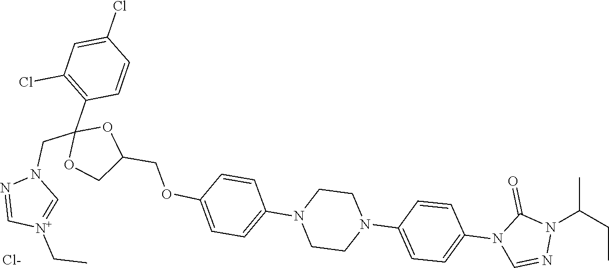 [SMILES: CCC(C)n1ncn(-c2ccc(N3CCN(c4ccc(OCC5COC(Cn6c[n+](CC)cn6)(c6ccc(Cl)cc6Cl)O5)cc4)CC3)cc2)c1=O ; [Cl-]] |
| Compound 3 | 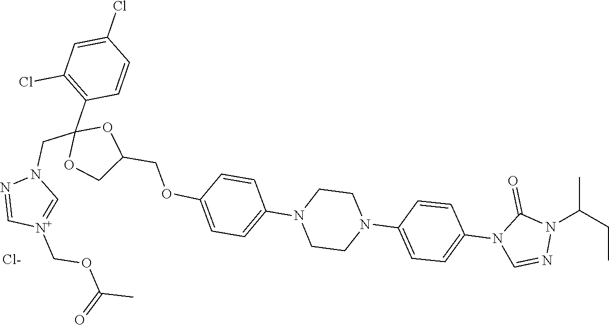 [SMILES: CCC(C)n1ncn(-c2ccc(N3CCN(c4ccc(OCC5COC(Cn6c[n+](COC(C)=O)cn6)(c6ccc(Cl)cc6Cl)O5)cc4)CC3)cc2)c1=O ; [Cl-]] |
| Compound 4 | 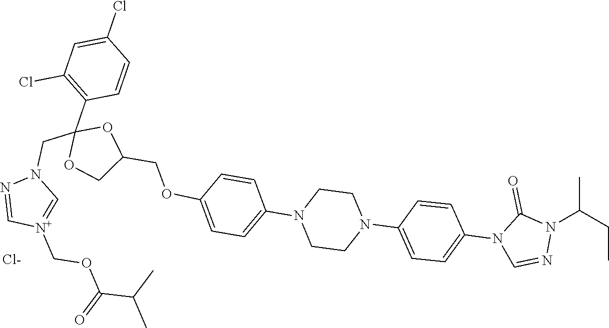 [SMILES: CCC(C)n1ncn(-c2ccc(N3CCN(c4ccc(OCC5COC(Cn6c[n+](COC(=O)C(C)C)cn6)(c6ccc(Cl)cc6Cl)O5)cc4)CC3)cc2)c1=O ; [Cl-]] |
| Compound 5 | 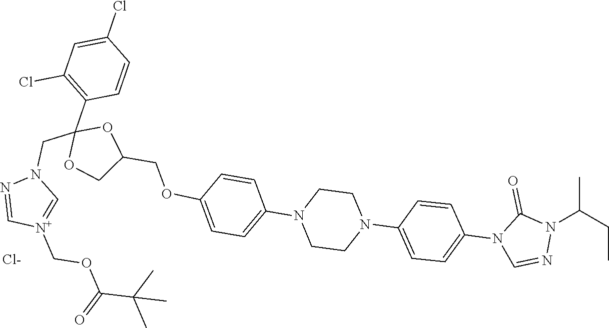 [SMILES: CCC(C)n1ncn(-c2ccc(N3CCN(c4ccc(OCC5COC(Cn6c[n+](COC(=O)C(C)(C)C)cn6)(c6ccc(Cl)cc6Cl)O5)cc4)CC3)cc2)c1=O ; [Cl-]] |
| Compound 6 | 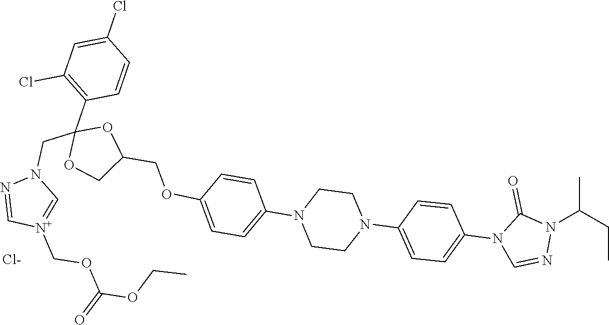 [SMILES: CCOC(=O)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]] |
| Compound 7 | 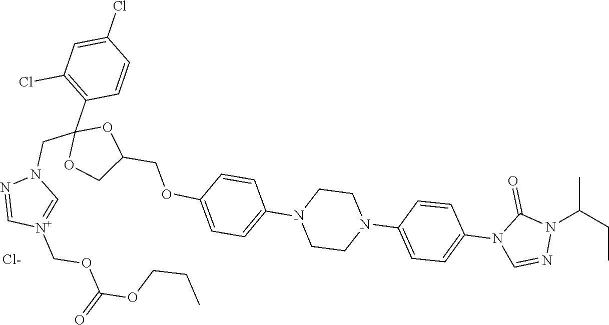 [SMILES: CCCOC(=O)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]] |
| Compound 8 | 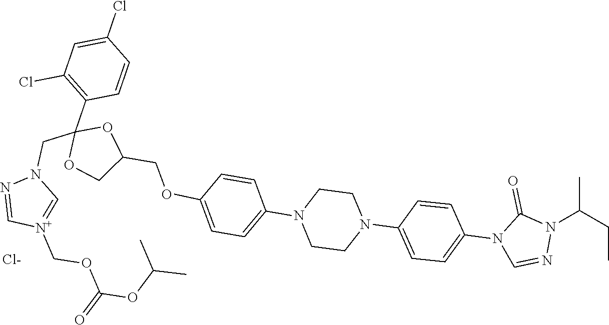 [SMILES: CCC(C)n1ncn(-c2ccc(N3CCN(c4ccc(OCC5COC(Cn6c[n+](COC(=O)OC(C)C)cn6)(c6ccc(Cl)cc6Cl)O5)cc4)CC3)cc2)c1=O ; [Cl-]] |
| Compound 9 | 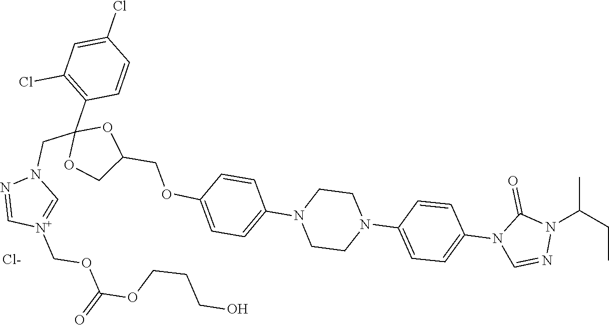 [SMILES: CCC(C)n1ncn(-c2ccc(N3CCN(c4ccc(OCC5COC(Cn6c[n+](COC(=O)OCCCO)cn6)(c6ccc(Cl)cc6Cl)O5)cc4)CC3)cc2)c1=O ; [Cl-]] |
| Compound 10 | 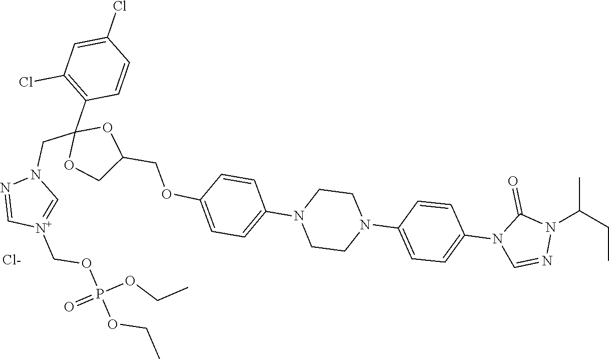 [SMILES: CCOP(=O)(OCC)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]] |

In an embodiment, the compound of Formula (I) has the structure of Formula A, or a pharmaceutically acceptable salt or solvent thereof,

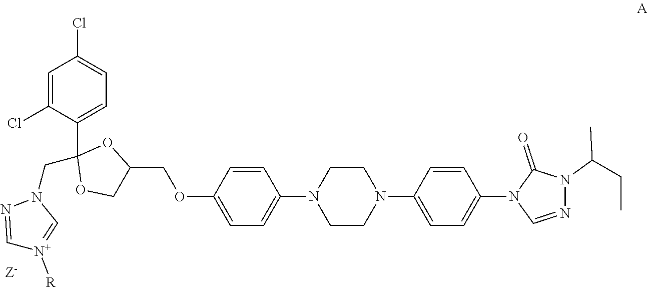

wherein X, U, V, and W are omitted, R is a C₁₋₅ alkyl and Z⁻ represents F⁻, Cl⁻, Br⁻, I⁻, or another pharmaceutically acceptable anion.

In another, the compound of Formula (I) has the structure of Formula B, or a pharmaceutically acceptable salt or solvent thereof,

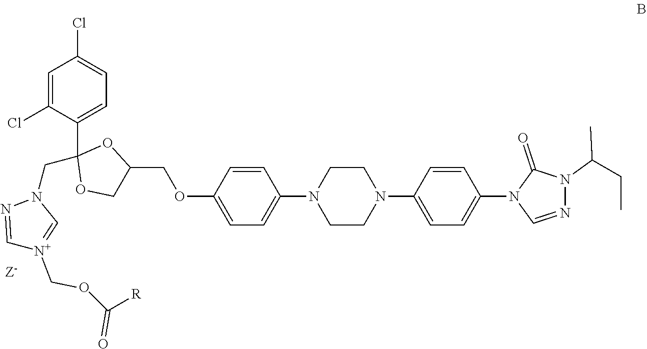

wherein X is —CH₂—, W is omitted and X, U, V, and R together form an ester, wherein R is a C₁₋₅ alkyl and Z⁻ represents F⁻, Cl⁻, Br⁻, I⁻, or another pharmaceutically acceptable anion.

In another embodiment, the compound of Formula (I) has the structure of Formula C, or a pharmaceutically acceptable salt or solvent thereof,

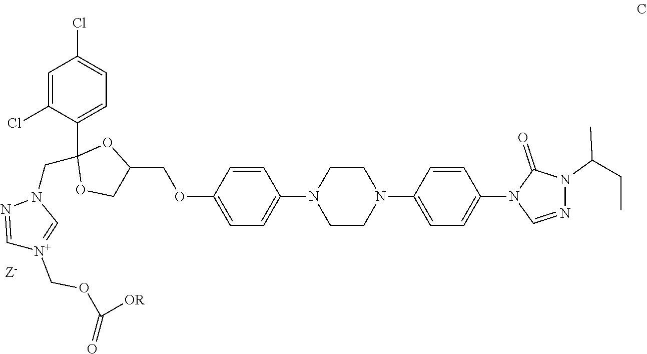

wherein X is —CH₂— and X, U, V, W, and R together form a carbonate ester, wherein R is a C₁₋₅ alkyl or hydroxyl-C₁-C₅ alkyl and Z⁻ represents F⁻, Cl⁻, Br⁻, I⁻, or another pharmaceutically acceptable anion.

In another embodiment, the compound of Formula (I) has the structure of Formula D, or a pharmaceutically acceptable salt or solvent thereof,

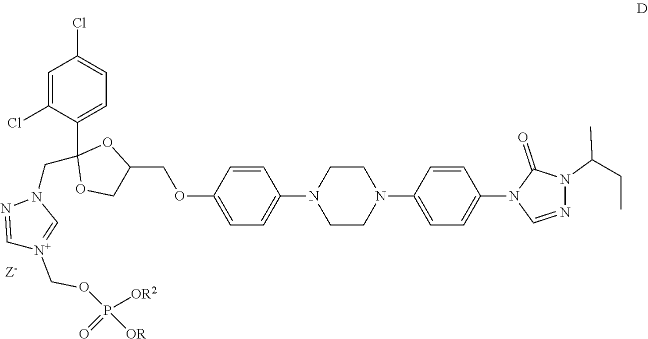

wherein X is —CH₂—, W is omitted, and U and V together form a phosphate, wherein R and R² are each independently a C₁₋₅ alkyl or C₁₋₅ hydroxy alkyl.

The disclosure also provides pharmaceutically acceptable compositions which comprise a therapeutically effective amount of one or more compounds of Formula (I), formulated together with one or more pharmaceutically acceptable carriers (additives) and/or diluents, and optionally, one or more additional therapeutic agents described above if needed. The compounds of this disclosure can be administered by any suitable means, for example, orally, as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. The pharmaceutical composition of the present disclosure can also be prepared as liposomes and nanoparticles. The composition in inhaler, for example, can be more effective than an oral dosage, with fewer side effects for all forms of lung fibrosis. Lower doses can be used, reducing the overall side effect burden.

Embodiments of the invention can include administration orally, sublingually, bucally, parenterally, by infusion, nasally, topically, or rectally, wherein the administration involves administration of one or more compounds of Formula (I) such as, for example, administration of a compound of Formula A, B, C, or D.

Embodiments of the invention can include administration orally, sublingually, bucally, parenterally, by infusion, nasally, topically, or rectally, wherein the administration involves administration of one or more compounds of Formula (I) such as, for example, administration of a compound of Formula A and B, A and C, A and D, B and C, B and D, or C and D.

Embodiments of the invention can include administration orally, sublingually, bucally, parenterally, by infusion, nasally, topically, or rectally, wherein the administration involves administration of one or more compounds of Formula (I) such as, for example, administration of a compound of Formula A, B, and C; A, B, and D; A, C, and D; and B, C, and D.

Embodiments of the invention can include administration orally, sublingually, bucally, parenterally, by infusion, nasally, topically, or rectally, wherein the administration involves administration of one or more compounds of Formula (I) such as, for example, administration of a compound of Formula A, B, C, and D.

The dosage regimen for the pharmaceutical compositions of the disclosure will, of course, vary depending upon known factors, such as the pharmacodynamic characteristics of the particular agents and the mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. In some embodiments, the dosage form may provide a dosage of between 10 to 1400 mg, such as between 10 to 1200 mg, or 10 to 1000 mg, or 10 to 800 mg, or 10 to 600 mg, or 10 to 500 mg, or 10 to 400 mg, or 10 to 200 mg, or 10 to 100 mg, or 10 to 50 mg of one or several of these compounds. The dosage form may also be formulated to provide a daily dosage in the range of 1-20 mg (e.g., 1-19 mg, or 1-18 mg, or 1-17 mg, or 1-16 mg, or 1-15 mg, or 1-14 mg, or 1-13 mg, or 1-12 mg, or 1-11 mg, or 1-10 mg, or 1-9 mg, or 1-8 mg, or 1-7 mg, or 1-6 mg, or 1-5 mg) per kg of body weight.

The pharmaceutical composition of this disclosure may be administered in a single daily dose, or the total daily dosage may be administered in divided doses of two, three, or four times daily.

Reference is made to U.S. Pat. No. 10,463,740 entitled “Itraconazole compositions and dosage forms, and methods of using the same”, incorporated herein by reference.

A composition comprising one or more compounds of Formula (I) can comprise therapeutically effective amount of one or more compounds of Formula (I) that delivers a therapeutically effective amount of itraconazole; for instance a composition comprising one or more compounds of Formula (I) can comprise a therapeutically effective amount of one or more compounds of Formula (I) that delivers up to about 75 mg of itraconazole, e.g., up to about 75 mg of one or more compounds of Formula (I), or a therapeutically amount of one or more compounds of Formula (I) that delivers up to about 50 mg to about 65 mg of itraconazole, e.g., about 50 mg to about 65 mg of one or more compounds of Formula (I). Particular compositions can comprise therapeutically effective amount of one or more compounds of Formula (I) that delivers a therapeutically effective amount of itraconazole of about 50 mg, e.g., about 50 mg of one or more compounds of Formula (I). Other particular compositions can comprise therapeutically effective amount of one or more compounds of Formula (I) that delivers a therapeutically effective amount of itraconazole of about 65 mg, e.g., about 65 mg of one or more compounds of Formula (I).

When administered to a subject in the fed state, a composition, such as any described herein, and particular a composition that delivers about 50 mg of itraconazole (e.g., about 50 mg of one or more compounds of Formula (I)), can exhibit certain pharmacokinetic parameters. For example, when administered to a subject in the fed state, the composition can exhibit an AUC₀₋ₜ of itraconazole of about 440 ng hr/mL or higher, such as from about 440 ng hr/mL to about 740 ng hr/mL, about 440 ng hr/mL to about 700 ng hr/mL, or about 448 ng hr/mL to about 676 ng hr/mL. In particular, when administered to a subject in the fed state, the composition that delivers about 50 mg of itraconazole (e.g., about 50 mg of one or more compounds of Formula (I)) can exhibit an AUC₀₋ₜ of itraconazole from about 475 to about 625 ng hr/mL. Even more particularly, when administered to a subject in the fed state, the composition that delivers about 50 mg of itraconazole (e.g., about 50 mg of one or more compounds of Formula (I)) can exhibit an AUC₀₋ₜ of itraconazole from about 500 to about 600 ng hr/mL. Thus, the ratio of AUC₀₋ₜ (in ng hr/mL) of itraconazole administered in a fed state to itraconazole delivered by one or more compounds of Formula (I) mass (in mg) can be about 8.8 or higher, such as from about 8.8 to about 14.8, about 8.8 to about 14.0, about 9.0 to about 13.6, about 9.5 to about 12.5, or about 10.0 to about 12.0 (e.g., the ratio of AUC₀₋ₜ (in ng hr/mL) of itraconazole administered in a fed state to one or more compounds of Formula (i) mass (in mg) can be about 8.8 or higher, such as from about 8.8 to about 14.8, about 8.8 to about 14.0, about 9.0 to about 13.6, about 9.5 to about 12.5, or about 10.0 to about 12.0.

A composition, including any dosage form described herein, and particularly a composition comprising one or more compounds of Formula (I) that delivers about 50 mg of itraconazole (e.g., about 50 mg of one or more compound of Formula (I)), can also have a particular AUC₀₋ₜ of itraconazole when the composition is administered to a subject in the fasted state. For example, when the composition is administered to a subject in the fasted state, the AUC₀₋ₜ of itraconazole can be about 350 ng hr/mL or higher, such as from about 350 to about 620 ng hr/mL, about 355 to about 550 ng hr/mL, or about 359 to about 534 ng hr/mL. In particular, when administered to a subject in the fasted state, the AUC₀₋ₜ of itraconazole can be from about 375 to about 515 ng hr/mL. Even more particularly, when the composition is administered to a subject in the fasted state, the AUC₀₋ₜ of itraconazole can be from about 400 to about 500 ng hr/mL. Thus, the ratio of AUC₀₋ₜ (in ng hr/mL) of itraconazole when one or more compounds of Formula (I) are administered to deliver about 50 mg of itraconazole to a subject in the fasted state to mass of itraconazole or to mass of one or more compounds of Formula (I) (in mg) can be about 7.0 or higher, such as from about 7.0 to about 12.4, about 7.1 to about 11.0, about 7.0 to about 10.7, about 7.5 to about 10.3, or about 8.0 to about 10.0.

A composition, including any described herein, and particularly a composition comprising an amount of one or more compounds of Formula (I) that delivers about 50 mg of itraconazole (e.g., about 50 mg of of one or more compounds of Formula (I)), can have a particular AUC_{∞} of itraconazole when the composition is administered to a subject in the fed state. For example, the AUC_{∞}, of itraconazole, when the composition is administered to a subject in the fed state, can be about 575 ng hr/mL or higher, such as about 590 to about 750 ng hr/mL. Particularly, the AUC_{∞}, of itraconazole, when the composition is administered to a subject in the fed state, can be about 591 to about 736 ng hr/mL, such as about 600 to about 725 ng hr/m. Even more particularly, the AUC_{∞}, of itraconazole, when the composition is administered to a subject in the fed state, can be about 625 to about 700 ng hr/mL. Thus, the ratio of AUC_{∞} of itraconazole when one or more compounds of Formula (I) that delivers about 50 mg of itraconazole is administered to a subject in the fed state (in ng hr/mL) to mass of itraconazole or one or more compounds of Formula (I) (in mg) can be about 11.5 or higher, such as about 11.8 to about 15, about 12 to about 14.5, or about 12.5 to about 14.

A composition, including any described herein, and particularly a composition comprising one or more compounds of Formula (I) and delivering about 50 mg of itraconazole or comprising about 50 mg of one or more compounds of Formula (I), can have a particular AUC_{∞} of itraconazole when the composition administered to a subject in the fasted state. For example, the AUC_{∞}, of itraconazole when administered to a subject in the fasted state, can be about 500 ng hr/mL or higher, such as about 521 ng hr/mL to about 611 ng hr/mL. Particularly, the AUC_{∞}, of itraconazole, when the composition is administered to a subject in the fasted state, can be about 550 ng hr/mL to about 600 ng hr/mL. Thus, the ratio of AUC_{∞} of itraconazole, when the composition is administered to a subject in the fasted state (in ng hr/mL) to mass of itraconazole or one or more compounds of Formula (I) (in mg) can be about 10 or higher, such as about 10.4 to about 12.22, or about 11.0 to about 12.0.

A composition, including any described herein, and particularly a composition in any dosage form comprising one or more compounds of Formula (I) and delivering about 50 mg of itraconazole e.g., a composition in any dosage form about 50 mg of one or more compounds of Formula (I), can have a particular Cₘₐₓ of itraconazole when administered to a subject in the fed state. For example, when the composition is administered to a subject in the fed state, the Cₘₐₓ of itraconazole can be about 60 ng/mL or higher, such as from about 60 to about 75 ng/mL or about 63 to about 75 ng/mL. In particular, when the composition is administered to a subject in the fed state, the Cₘₐₓ of itraconazole can be from about 65 to about 70 ng/mL.

A composition, including any described herein, and particularly a composition in any dosage form comprising one or more compounds of Formula (I) and delivering about 50 mg of itraconazole e.g., a composition in any dosage form comprising about 50 mg of one or more compounds of Formula (I), can have a particular Cₘₐₓ of itraconazole when administered to a subject in the fasted state. For example, when the composition is administered to a subject in the fasted state, the Cₘₐₓ of itraconazole can be about 30 ng/mL or higher, such as about 30 ng/mL to about 60 ng/mL or about 32 ng/mL to about 55 ng/mL. In particular, when administered the composition is administered to a subject in the fasted state, the Cₘₐₓ of itraconazole can be from about 37 ng/mL to about 52 ng/mL or about 35 ng/mL to about 50 ng/mL. More particularly, when the composition is administered to a subject in the fasted state, the Cₘₐₓ of itraconazole can be from about 40 ng/mL to about 50 ng/mL or about 42 ng/mL to about 50 ng/mL.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular AUC₀₋ₜ of itraconazole when administered to a subject in the fed state. For example, the AUC₀₋ₜ, of itraconazole when the composition is administered to a subject in the fed state, can be about 650 ng hr/mL or greater, such as about 650 to about 1200 ng hr/mL or about 671 to about 1172 ng hr/mL. Particularly, the AUC₀₋ₜ, of itraconazole when the composition is administered to a subject in the fed state, can be about 700 to about 950 ng hr/mL. Even more particularly, the AUC₀₋ₜ, of itraconazole, when the compositions administered to a subject in the fed state, can be about 750 to about 850 ng hr/mL. Thus, the ratio of AUC₀₋ₜ (in ng hr/mL) of itraconazole when the composition is administered to a subject in the fed state to mass of itraconazole or of one or more compounds of Formula (I) (in mg) can be about 10.0 or higher, such as about 10.3 to about 18.0, about 10.8 to about 14.6, or about 11.5 to about 13.0.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular AUC₀₋ₜ of itraconazole when administered to a subject in the fasted state. For example, the AUC₀₋ₜ, of itraconazole when the composition is administered to a subject in the fasted state, can be about 450 ng hr/mL or greater, such as about 450 to about 900 ng hr/mL, about 485 to about 900 ng hr/mL, or about 500 to about 885 ng hr/mL. Particularly, the AUC₀₋ₜ, of itraconazole when the composition is administered to a subject in the fasted state, can be about 525 to about 725 ng hr/mL. Even more particularly, the AUC₀₋ₜ, of itraconazole when the composition is administered to a subject in the fasted state, can be about 600 to about 700 ng hr/mL. Thus, the ratio of AUC₀₋ₜ (in ng hr/mL) of itraconazole when the composition is administered to a subject in the fasted state to the mass of itraconazole or of one or more compounds of Formula (I) (in mg) can be about 7.5 or greater, such as about 7.5 to about 13.6, about 7.7 to about 13.6, about 801 to about 11.2, or about 9.2 to about 10.8.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular AUC_{∞} of itraconazole when administered to a subject in the fed state. For example, the AUC_{∞}, of itraconazole, when the composition is administered to a subject in the fed state, can be about 800 ng hr/mL or greater, such as about 811 ng hr/mL to about 1,400 ng hr/mL. In particular, the AUC_{∞}, of itraconazole, when the composition is administered to a subject in the fed state, can be about 850 ng hr/mL to about 1,200 ng hr/mL. Even more particularly, the AUC_{∞}, of itraconazole, when the composition is administered to a subject in the fed state, can be about 900 ng hr/mL to about 1,000 ng hr/mL, or about 850 to about 950 ng hr/mL. Thus, the ratio of the AUC_{∞} (in ng hr/mL) of itraconazole when the composition is administered to a subject in the fed state to the mass of itraconazole or of one or more compounds of Formula (I) (in mg) can be about 12.3 or greater, such as, about 12.3 to about 21.5, about 12.5 to about 21.5, about 13.1 to about 18.5, about 13.9 to about 15.4, or about 13.1 to about 14.6.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular AUC_{∞} of itraconazole when administered to a subject in the fasted state. For example, the AUC_{∞}, of itraconazole, when the compositions is administered to a subject in the fasted state, can be about 600 ng hr/mL or greater, such as about 610 ng hr/mL to about 1,050 ng hr/mL. In particular, the AUC_{∞}, of itraconazole, when the composition is administered to a subject in the fasted state, can be about 640 ng hr/mL to about 900 ng hr/mL. Even more particularly, the AUC_{∞}, of itraconazole, when the composition is administered to a subject in the fasted state, can be about 675 ng hr/mL to about 750 ng hr/mL, or about 625 to about 800 ng hr/mL. Thus, the ratio of AUC_{∞} (in ng hr/mL) of itraconazole when the composition is administered to a subject in the fasted state to the mass of itraconazole or one or more compounds of Formula (I) (in mg) can be about 9.2 or greater, such as about 9.2 to about 16.2, about 9.4 to about 16.2, about 9.8 to about 13.8, about 10.4 to about 12.3, or about 9.6 to about 11.5.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular Cₘₐₓ of itraconazole, when administered to a subject in the fed state, can have a Cₘₐₓ of itraconazole of about 65 ng/mL or higher, such as about 85 ng/mL to about 100 ng/mL. Particularly, when the composition is administered to a subject in the fed state, the Cₘₐₓ of itraconazole can be about 70 ng/mL to about 80 ng/mL. Thus the ratio of Cₘₐₓ (in ng/mL) of itraconazole when the composition is administered to a subject in the fed state to the mass of itraconazole or of one or more compounds of Formula (I) (in mg) can be about 1.00 or greater, such as about 1.00 to about 1.54 about 1.31 to about 1.54, or about 1.08 to about 1.23.

A composition, including any described herein, and particularly a composition in any dosage form comprising one or more compounds of Formula (I) and delivering about 65 mg of itraconazole e.g., a composition in any dosage form comprising about 65 mg of one or more compounds of Formula (I), can have a particular Cₘₐₓ of itraconazole when administered to a subject in the fasted state of about 35 ng/mL or higher, such as about 35 ng/mL to about 70 ng/mL. Particularly, when the composition is administered to a subject in the fasted state, the Cₘₐₓ of itraconazole can be about 40 ng/mL to about 65 ng/mL. Thus the ratio of Cₘₐₓ (in ng/mL) of itraconazole when the composition is administered to a subject in the fasted state to the mass of itraconazole or of one or more compounds of Formula (I) (in mg) can be about 0.54 or greater, such as about 0.54 to about 1.08, or about 0.62 to about 1.00.

The present composition comprising one or more compounds of Formula (I) can also comprise one or more excipients. The excipients can include one or more of waxes, polymers, binders, fillers, disintegrants, glidants, and the like. The polymers can include any pharmaceutically acceptable polymer, such as one or more hydrophilic polymers; one or more non-gelling polymers; one or more acid-resistant polymers and enteric polymers; one or more osmopolymers; one or more film-forming, water insoluble polymers; one or more film-forming, water soluble polymers; or combinations thereof. The waxes can include one or more of beeswax, spermaceti, lanolin, carnauba wax, candelilla wax, ouricury wax, sugercane wax, retamo wax, jojoba oil, epicuticula waxes, paraffin, montan wax, waxes produced from cracking polyethylene, microcrystalline wax, petroleum jelly, and the like.

Binders can include any one or more of saccharides, such as sucrose, lactose, mannose, trehaolse, fructose, starches, cellulose, microcrystalline cellulose, methyl cellulose, ethyl cellulose, hydroxypropyl cellulose, hydroxypropyl methylcellulose, and the like, gelatin, polyvinylpyrrolidone, polyethylene glycol, and the like.

Disintegrants can include one or more of crospovidone, croscarmellose, such as crosscarmellose sodium, polyvinylpyrrolidone, methyl cellulose, microcrystalline cellulose, lower alkyl-substituted hydroxypropyl cellulose, such as hydroxypropyl methyl cellulose and hydroxypropyl ethyl cellulose, starch, pregelatinised starch, sodium alginate, and sodium starch glycolate, for example, sodium starch glycolate.

Fillers can include one or more of cellulose, microcrystalline cellulose, dibasic calcium phosphate, monobasic calcium phosphate, lactose, sucrose, glucose, mannitol, sorbitol, calcium carbonate, and the like.

Polymers can include any pharmaceutically acceptable polymer. The polymer can be formulated with the active compound (e.g., one or more compounds of Formula (I)) and one or more additional excipients in various forms. For example, the present composition may be formulated to a matrix system, an osmotic delivery system, or a multiparticulate system. As used herein, the term “matrix” denotes a homogeneous solid mixture composed of evenly dispersed ingredients throughout. In one embodiment, the matrix system is a solid solution or solid dispersion as described herein.

In an embodiment the delivery system for one or more compounds of Formula (I) can be an osmotic delivery system, whereby the composition comprises a release rate controlling membrane disposed over a pull layer and an osmotic push layer, wherein the pull layer comprises one or more compounds of Formula (I), and the release rate controlling membrane has an orifice immediately adjacent to the pull layer. The pull layer further optionally comprises a release rate controlling polymer and/or a pharmaceutically acceptable excipient. The release rate controlling membrane is a semipermeable wall that surrounds the pull layer and the osmotic push layer. The wall is permeable to the passage of fluid and has an orifice which allows passage of one or more compounds of Formula (I), from inside of the wall to outside. Upon being exposed to biological or other fluids, the semipermeable wall allows permeation of the fluids through the wall causing expansion of the osmotic push layer, and consequently the osmotic push layer pushes the pull layer through the orifice. The release rate of one or more compounds of Formula (I), is determined by the permeability of the wall and the osmotic pressure gradient across the wall. In one embodiment, the osmotic push layer comprises an osmopolymer. In an embodiment, the pull layer further comprises an osmagent, also known as osmotically effective solutes. The osmagent can be any compound, inorganic or organic, that exhibit an osmotic pressure gradient across an external fluid across the semipermeable wall.

Certain examples of a multiparticulate delivery system and the manufacturing thereof that can be used in the practice of this invention to deliver one or more compounds of Formula (I) are described in detail in Lu, Int. J. Pharm., 1994, 112, pages 117-124, the content of which is herein incorporated by reference in its entirety. In an embodiment, the composition comprises one or more particles and each of the particles comprises an active core comprising one or more compounds of Formula (I); and a release rate controlling polymer disposed over the core. In another embodiment, the composition comprises one or more particles and each of the particles comprises an inert core, an active layer comprising one or more compounds of Formula (I) disposed over the inert core, and a release rate controlling polymer disposed over the active layer. In another embodiment, the composition comprises an inert core, and a coating disposed over the inert core, wherein the coating comprises one or more compounds of Formula (I). Any of the active core, the inert core, the active layer, the coating, or the coating formed by the release rate controlling polymer disposed over the active layer may optionally further comprise a pharmaceutically acceptable excipient. In an embodiment of the multiparticulate delivery system, the release rate controlling polymer comprises a film-forming, water insoluble polymer in combination with a film-forming, water soluble polymer. The ratio between the water insoluble polymer and the water soluble polymer can be adjusted depending on the intended drug release profile.

“Hydrophilic polymer” refers to a polymer having a strong affinity for water and tending to dissolve in, mix with, or be wetted by water. Examples of the hydrophilic polymer include, but are not limited to polyethylene oxide, hydroxypropyl cellulose, hydroxypropyl methyl cellulose, hydroxyethyl cellulose, sodium carboxymethylcellulose, calcium carboxymethyl cellulose, methyl cellulose, polyacrylic acid, maltodextrin, pre-gelatinized starch, guar gum, sodium alginate, polyvinyl alcohol, chitosan, locust bean gum, amylase, any other water-swelling polymer, and a combination thereof.

By “non-gelling polymer”, it is meant a polymer that only swells slightly or does not swell to form a gel when exposed to an aqueous medium. Exemplary non-gelling polymers include cellulose acetate phthalate (e.g., powder: pH 6.2, available from Eastman Chemical Co. as C-A-P; Dispersion: pH: 6.0, available from FMC BioPolymer as AquaCoat® CPD), cellulose acetate succinate (e.g., LF: pH 5.5; MF: pH 6.0; HF: pH 6.8; LG; pH 5.5; MG: pH 6.0; HG: 6.8, F grades are an aqueous dispersion and G grades are from solvent available from Shin-Etsu under the trade name AQOAT®), hypromellose phthalate (HPMCP) (e.g., Grade HP-50: pH 5.0; Grade HP-55: pH 5.5 available from Shin-Etsu), hypromellose acetate succinate (HPMCAS), polyvinylacetate phthalate (e.g., aqueous dispersion: pH 5.0; Powder: pH 5.0 available from Colorcon, the aqueous dispersion under the trade name Sureteric® and the powder under the trade name Opadry®. Enteric), hydroxyethyl cellulose phthalate, cellulose acetate maleate, cellulose acetate trimellitate, cellulose acetate butyrate, cellulose acetate propionate, methacrylic acid-methyl methacylate co-polymers (e.g., Type A: pH 6.0; Type B: pH 7.0 both available from Degussa/Evonik with the trade names EUDRAGIT® L 100 for Type A and EUDRAGIT® S 100 for Type B), methacrylic acid-ethylacrylate co-polymers (available under the trade name EUDRAGIT® L, e.g., L100-55), methacrylic acid-methyl acrylate-methyl methacrylate co-polymers (available under the trade name EUDRAGIT® FS-30D for delivery above pH 7.0), and the like or combinations comprising at least one of the foregoing. Methacrylic acid-methyl methacylate co-polymers, methacrylic acid-ethylacrylate co-polymers, and/or methacrylic acid-methyl acrylate-methyl methacrylate co-polymers are also known as polymethacrylates as described in the Handbook of Pharmaceutical Excipients, 2006, the Fifth Edition, edited by Raymond C Rowe, Paul J. Sheskey, and Sian C Owen, pages 553 to 560, the content of which is incorporated by references in its entirety. EUDRAGIT® is a trademark of Evonik Industries. The specifications for various EUDRAGIT® products including the above-mentioned ones can be found in the manufacture's product manual or on the website for the corresponding EUDRAGIT® product, the content of which is incorporated by references in its entirety.

The osmopolymers are typically hydrophilic polymers and interact with water and aqueous biological fluids and swell or expand to push a drug composition through the orifice. The osmopolymers exhibit the ability to swell in water and retain a significant portion of the imbibed water within the polymer structure. The osmopolymers may swell or expand to a very high degree. The osmopolymers can be noncross-linked or cross-linked. The swellable, hydrophilic polymers may be lightly cross-linked, such as cross-links being formed by covalent or ionic bonds. The osmopolymers can be of plant, animal or synthetic origin. Hydrophilic polymers suitable for the present purpose include, but are not limited to poly(hydroxyalkylmethacrylate) having a molecular weight of from 30,000 to 5,000,000; poly(vinylpyrrolidone) having molecular weight of from 10,000 to 360,000; anionic and cationic hydrogels; polyelectrolyte complexes, poly(vinyl alcohol) having a low acetate residual, cross-linked with glyoxal, formaldehyde, or glutaraldehyde and having a degree of polymerization from 200 to 30,000; a mixture of methyl cellulose, cross-linked agar and carboxymethyl cellulose; a water insoluble, water swellable copolymer reduced by forming a dispersion of finely divided copolymer of maleic anhydride with styrene, ethylene, propylene, butylene or isobutylene cross-linked with from 0.00001 to about 0.5 moles of polyunsaturated cross-linking agent per mole of maleic anhydride in the copolymer; water swellable polymers of N-vinyl lactams, and the like. Other osmopolymers include hydrogel polymers, such as Carbopol® (acrylic acid-based polymers crosslinked with polyalkylene polyethers) and the sodium salt thereof; acidic carboxy polymers generally having a molecular weight of 450,000 to 4,000,000 and their metal salts; Polyox™; polyethylene oxide polymers having a molecular weight of 100,000 to 7,500,000.

Examples of the film-forming, water insoluble polymer include, but are not limited to ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride) or polyurethane, or any other water insoluble polymer, or mixtures thereof.

Examples of the film-forming, water soluble polymer include, but are not limited to polyvinyl alcohol, polyvinylpyrrolidone, methyl cellulose, hydroxypropyl cellulose, hydroxypropylmethyl cellulose and polyethylene glycol, Pluronic® F108, Pluronic® F127, Pluronic® F68 or mixtures thereof.

The present invention in an embodiment provides a composition comprising one or more compounds of Formula (I) formulated in or into a matrix system. The composition comprising one or more compounds of Formula (I) can comprise a solid solution or solid dispersion, for example, a solid dispersion, of one or more compounds of Formula (I) in a pharmaceutically acceptable carrier. The pharmaceutically acceptable carrier can be a polymer. Exemplary polymers include acid-resistant polymers and enteric polymers, although other polymers can also be used. Acid-resistant polymers can include polymers that are insoluble in water at any pH and polymers that are insoluble in water at an acidic pH, such as enteric polymers. Exemplary acid-resistant polymers include hydroxypropyl methylcellulose phthalate, polyvinyl acetate phthalate, hydroxypropyl methylcellulose acetate, such as hydroxypropyl methylcellulose acetate succinate, alginate, poly(meth)acrylic acid homopolymers and copolymers, carbomers, carboxymethyl cellulose, carboxymethyl cellulose, methacrylic acid copolymers, shellac, cellulose acetate phthalate, hydroxypropyl cellulose acetate phthalate, cellulose acetate terephthalate, methyl cellulose acetate phthalate, cellulose acetate isophthalate, cellulose acetate trimellitate, EUDRAGIT® polymers (copolymers of one or more of poly(meth)acrylates, poly(meth)acrylic esters, and poly(meth)acrylamides), and the like. A particular exemplary acid-resistant polymer is hydroxypropyl methylcellulose phthalate.

Exemplary enteric polymers include one or more of hydroxypropyl methylcellulose phthalate; polyvinyl acetate phthalate; hydroxypropylmethylcellulose acetate succinate; alginate; carbomer; carboxymethyl cellulose; methacrylic acid copolymer; shellac; cellulose acetate phthalate; starch glycolate; polacrylin; cellulose acetate phthalate; methyl cellulose acetate phthalate; hydroxypropylcellulose acetate phthalate; cellulose acetate terephthalate; cellulose acetate isophthalate; and cellulose acetate trimellitate. A particular enteric polymer is hydroxypropyl methylcellulose phthalate, which is commercially available from Shin-Etsu Chemical Industry Co Ltd under the trade names HP-50, HP-55, and HP-55S.

A composition comprising one or more compounds of Formula (I) can comprise a solid solution or solid dispersion, for example, a solid dispersion, of one or more compounds of Formula (I) in a pharmaceutically acceptable carrier. The pharmaceutically acceptable carrier can be a polymer, such as an acid-resistant polymer or an enteric polymer, particularly the acid-resistant polymers discussed herein, or the enteric polymers discussed herein, and, for example, more particularly hydroxypropyl methylcellulose phthalate, which is commercially available from Shin-Etsu Chemical Industry Co Ltd under the trade names HP-50, HP-55, and HP-55S.

The solid solution or solid dispersion can be made by methods known in the art, for example, by methods disclosed in U.S. Pat. No. 6,881,745, which is hereby incorporated by reference in its entirety and for all purposes. For example, a solid solution or solid dispersion can be made by dissolving or dispersing the pharmaceutically acceptable carrier and the one or more compounds of Formula (I) in a suitable solvent and then removing the solvent. The suitable solvent can be, for example, one or more of methylene chloride, chloroform, ethanol, methanol, propan-2-ol, ethyl acetate, acetone, water, and mixtures thereof. A particular solvent is methylene chloride.

Removing the solvent can be accomplished by evaporation, spray drying, lyophilizing, and the like. Removing the solvent can also be accomplished by allowing the one or more compounds of Formula (I) and pharmaceutically acceptable carrier to co-precipitate or co-crystallize out of solution, followed by one or more of filtration, decanting, centrifuging, and the like.

Other methods of forming solid solutions or solid dispersions include co-grinding, melt extrusion, freeze drying, rotary evaporation, and other solvent removal processes.

When the one or more compounds of Formula (I) is in the form of a solid dispersion, the solid dispersion can be present in sufficient amounts to provide a therapeutically effective amount of one or more compounds of Formula (I) (which delivers a therapeutically effective amount of a itraconazole). The therapeutically effective amount of one or more compounds of Formula (I), which in the case of a salt, solvate, ester, or the like is measured by the amount of free compound(s) of the one or more compounds of Formula (I), can be an amount of one or more compounds of Formula (I) that delivers up to about 100 mg of itraconazole (e.g., up to about 100 mg of one or more compounds of Formula (I)), for example, an amount of one or more compounds of Formula (I) that delivers up to about 70 mg. Exemplary amounts of itraconazole to be delivered by by an amount of one or more compounds of Formula (I) for a single dosage form include an amount of one or more compounds of Formula (I) to deliver about 48 mg to about 68 mg of itraconazole, such as about 50 mg to about 65 mg of itraconazole, for instance about 50 mg to about 65 mg of itraconazole, for example, about 50 mg or about 65 mg of itraconazole (e.g., about 48-68 mg of one or more compounds of Formula (I), such as about 50 mg to about 65 mg of itraconazole, for instance about 50 mg to about 65 mg of itraconazole, for example, about 50 mg or about 65 mg).

The weight ratio of the one or more compounds of Formula (I) in the solid solution or solid dispersion to the pharmaceutically acceptable carrier, such as hydroxypropyl methylcellulose phthalate, can be from about 3:1 to about 1:20, such as about 3:1 to about 1:5, about 1:1 to about 1:3, or about 1:1.5, based on the weight of the one or more compounds of Formula (I). Thus, the pharmaceutically acceptable carrier, such as hydroxypropyl methylcellulose phthalate, can be present from about 15 mg to about 1,360 mg, for example, from about 15 mg to about 340 mg, about 48 to about 204 mg, or particularly about 72 to about 102 mg, for example, about 75 mg or about 97.5 mg.

The composition comprising a solid dispersion of one or more compounds of Formula (I) can further comprise one or more additional pharmaceutically acceptable excipients. When present, the one or more additional pharmaceutically acceptable excipients can be in the solid solution or dispersion, or outside of the solid solution or dispersion, such as admixed or blended with the solid solution or dispersion. The one or more additional pharmaceutically acceptable excipients can include one or more disintegrants, one or more diluents, one or more fillers, one or more colorants, one or more flavorants, one or more binders, one or more glidants, one or more lubricants, one or more surface active agents, and mixtures thereof.

Exemplary disintegrants include one or more of crospovidone, croscarmellose, such as crosscarmellose sodium, polyvinylpyrrolidone, methyl cellulose, microcrystalline cellulose, lower alkyl-substituted hydroxypropyl cellulose, such as hydroxypropyl methyl cellulose and hydroxypropyl ethyl cellulose, starch, pregelatinised starch, sodium alginate, and sodium starch glycolate, for example, sodium starch glycolate. The disintegrant is often present outside of solid solution or solid dispersion, and the weight ratio of the solid solution to solid dispersion can be from about 1:1 to about 1:10, such as about 2:1 to about 6:1, about 4:1 to about 5:1, for example, from about 4.2:1, although this is not required unless otherwise specified. For example, when the dosage form is a tablet, the dosage form can comprise from about 1% to about 25% of disintegrant by weight.

Exemplary colorants include one or more of titanium dioxide and food dyes.

Exemplary flavors include one or more of cinnamon oil, wintergreen oil, peppermint oil, bay oil, anise oil, eucalyptus oil, thyme oil, vanilla, such as tincture of vanilla, citrus oil, such as one or more of lemon, orange, lime, and grapefruit oil, and essences of fruits, such as essence of one or more of apple, banana, pear, peach, strawberry, raspberry, cherry, plum, pineapple, and apricot.

Exemplary lubricants include one or more of hydrogenated vegetable oil, magnesium stearate, sodium lauryl sulfate, magnesium lauryl sulfate, colloidal silica, and talc. In some examples, the lubricant is magnesium stearate. In other examples, the lubricant is colloidal silica. In yet other examples, the lubricant is a mixture of magnesium stearate and colloidal silica.

Exemplary glidants include one or more of silicon dioxide and talc.

Exemplary binders include one or more of microcrystalline cellulose, gelatin, sugars, such as one or more of mannitol, lactose, and cellulose, polyethylene glycol, gums, such as one or more of xanthan gum and guar gum, polyvinylpyrrolidone, pregelatinised starch, hydroxypropyl cellulose, and bydroxypropylmethylcellulose.

Exemplary diluants include one or more of lactose, such as one or more of lactose monohydrate, spray-dried lactose monohydrate, and anhydrous lactose, mannitol, xylitol, dextrose, sucrose, sorbitol, microcrystalline cellulose, starch, and calcium phosphate, such as dibasic calcium phosphate dihydrate.

Exemplary surface active agents include one or more of sodium lauryl sulfate, polyethylene glycol, and polysorbate 80.

The composition can be, for example, in the form of one or more dosage forms, such as one or more of a powder, sachet, tablet, capsule, pill, suppository, implant, wafer, cream, ointment, syrup, gel, suspension, and the like. When the dosage form is a capsule, the capsule shell can be a hard capsule shell, such as a gelatin shell, comprising the solid solution or solid dispersion of one or more compounds of Formula (I) and the pharmaceutically acceptable carrier. The capsule shell can also comprise one or more of the additional pharmaceutically acceptable excipients discussed above, although that is not required unless otherwise specified. The capsule shell can be a sufficient size to accommodate the contents of the capsule.

An exemplary capsule can be filled with a solid dispersion that comprises an amount of one or more compounds of Formula (I) to deliver about 50 mg of itraconazole, e.g., about 50 mg one or more compounds of Formula (I) (based on the weight of free compound(s) of the one or more compounds of Formula (I)) and about 75 mg hydroxypropyl methylcellulose phthalate, and, as additional pharmaceutical excipients not part of the dispersion, about 30 mg sodium starch glycolate, about 1 mg to about 2 mg colloidal silica, and about 1 mg to about 2 mg magnesium stearate. Another exemplary capsule can comprise an amount of one or more compounds of Formula (I) to deliver about 65 mg of itraconazole, e.g., about 65 mg one or more compounds of Formula (I) (based on the weight of free compound(s) of the one or more compounds of Formula (I)), about 97.5 mg hydroxypropyl methylcellulose phthalate, and, as additional pharmaceutical excipients not part of the dispersion, about 39 mg sodium starch glycolate, about 1.3 mg to about 2.6 mg colloidal silica, and about 1.3 mg to about 2.6 mg magnesium stearate.

When the dosage form is a tablet, the tablet can comprise the solid solution or solid dispersion of one or more compounds of Formula (I) and a pharmaceutically acceptable carrier such that the one or more compounds of Formula (I) is from about 1% to about 80%, such as about 5% to about 60%, by weight, of the tablet.

The tablet can also comprise one or more lubricant, such as the one or more lubricants discussed above. The one or more lubricant can be present from about 0.25% to about 10% by weight of the tablet.

The tablet can further comprise one or more disintegrants, such as one of more of the disintegrants discussed above. The one or more disintegrant can be present from about 1% to about 25% by weight of the tablet.

The tablet can further comprise one or more glidants, such as one or more of the glidants discussed above. The one or more glidants can be present from about 0.2% to about 1% by weight of the tablet.

The tablet can further comprise one or more surface active agents, such as one or more of the surface active agents discussed above. The one or more surface active agents can be present from about 0.2% to about 5% by weight of the tablet.

When the dosage form is a capsule, the capsule can comprise a therapeutically effective amount of one or more compounds of Formula (I), such as the amounts discussed herein or otherwise derivable from the disclosure herein. The remainder of the capsule can be filled with additional pharmaceutical excipients, such as those discussed herein.

The composition can be specially adapted to be administered in the fasted state. The terms “in the fasted state” and “under fasting conditions” are herein used interchangeably. Similarly, the terms “in the fed state” and “under fed conditions” are herein used interchangeably. The composition can also be administered in either the fed or fasted state. For example, the dosage form can have a reduced food effect. The reduced food effect can be a difference of less than about 35% between a AUC₀₋ₜ of itraconazole under fasting conditions and a AUC₀₋ₜ of itraconazole under fed conditions, for example a difference of less than about 33%, about 30%, about 27%, about 25%, about 23%, or about 20% between a AUC₀₋ₜ of itraconazole under fasting conditions and a AUC₀₋ₜ under fed conditions. In another example, the composition comprising one or more compounds of Formula (I) exhibits an absorption profile of itraconazole under fasting conditions which is substantially similar to the absorption profile of a reference dosage form of itraconazole under the proprietary name Sporanox® (the reference dosage form) under fed conditions. In particular, the substantial similarity is bioequivalence.

Without wishing to be bound by theory, it is believed that the use of a solid dispersion of one or more compounds of Formula (I) in an acid resistant pharmaceutically acceptable carrier can prevent the one or more compounds of Formula (I) from dissolving too fast in the gastric juice and subsequently precipitating out in the higher pH environment of the lower GI tract thereby increasing the consistency of the bioavailability of one or more compounds of Formula (I) and hence ultimately itraconazole.

The composition can be specially adapted to have an AUC of itraconazole with a reduced dose-to-dose intra-subject variability in the same subject. The reduced intra-subject variability can be with respect to the SPORANOX® dosage form of itraconazole. For example, the dosage form of a composition of the invention comprising one or more compounds of Formula (I) can have a reduced variability in the AUC₀₋ₜ, Cₘₐₓ, of itraconazole and/or Tₘₐₓ of itraconazole as compared to the reference dosage form, such as an intra-subject coefficient of variability under fed conditions for the AUC₀₋ₜ of itraconazole can be about 35% or less. As another example an intra-subject coefficient of variability under fed conditions for the AUC_{0-∞} of itraconazole can be about 35% or less.

Particular parameters of the composition of the invention comprising one or more compounds of Formula (I) can be defined with respect to the commercially available SPORANOX® (the “reference composition.”) For example, when the composition of the present invention is administered in the fed state, it can have one or more pharmacokinetic parameters that are therapeutically similar to those of reference composition when administered in the fed state. Such therapeutic similarity can be determined by a routine in vivo pharmacokinetic study to compare one or more pharmacokinetic parameters of the the two compositions. A pharmacokinetic parameter for the compositions can be measured in a single or multiple dose study using a replicate or a nonreplicate design. For example, the pharmacokinetic parameters for the present oral solid composition and for the reference composition can be measured in a single dose pharmacokinetic study using a two-period, two-sequence crossover design. Alternately, a four-period, replicate design crossover study may also be used. Single doses of the present composition and the reference composition are administered and blood or plasma levels of itraconazale are measured over time. Pharmacokinetic parameters characterizing rate and extent of one or more compounds of Formula (I) absorption are evaluated statistically. The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA). In one embodiment, two compositions (e.g. the present composition and the reference composition) or methods (e.g., dosing under fed versus fasted conditions) are therapeutically similar if the Confidence Interval (CI) range of 80% to 95% (e.g., including 90%) limits for a ratio of the geometric mean of logarithmic transformed AUC_{0-∞}, AUC₀₋ₜ, and/or Cₘₐₓ for the two compositions or two methods are about 0.70 to about 1.43; or about 0.75 to about 1.33; or about 0.80 to about 1.25.

In addition or in the alternative, the composition of the invention comprising one or more compounds of Formula (I) can be therapeutically equivalent to the reference composition (e.g., commercially available SPORANOX®). For example, administration of the composition of the invention over about the same time period as the reference composition (e.g., commercially available SPORANOX®) can produce a substantially similar therapeutic outcome.

The composition of the invention comprising one or more compounds of Formula (I) can be bioequivalent to the reference composition. For example, the composition of the invention can have 90% Confidence Interval (CI) limits for a ratio of the geometric mean of logarithmic transformed AUC_{0-∞}, AUC₀₋ₜ, and Cₘₐₓ of itraconazole for the composition is about 0.80 to about 1.25 of the reference composition (e.g., commercially available SPORANOX®). As another example, the composition of the invention comprising one or more compounds of Formula (I) can have 90% CI limits for a ratio of the geometric mean of logarithmic transformed AUC_{0-∞} and AUC₀₋ₜ of about 0.80 to about 1.25 of the reference composition (e.g., commercially available SPORANOX®).

The amount of one or more compounds of Formula (I) in the composition can be an amount to deliver an amount of itraconazole that is equal to or from about 50% to about 95%, e.g., about 50% to about 90%, or about 50% to about 85%, or about 50% to about 80% or about 50% to about 75%, or about 50% to about 70% or about 50% to about 65% by weight of the amount of itraconazole in the reference composition (e.g., commercially available SPORANOX®).

The composition of the invention comprising one or more compounds of Formula (I) can have an AUC₀₋ₜ of itraconazole that is about 0.70 to about 1.43 of that of the reference composition (e.g., commercially available SPORANOX®). The composition of the invention can have an AUC₀₋ₜ of itraconazole that is about 0.75 to about 1.33 of that of the reference composition (e.g., commercially available SPORANOX®). The composition can have a relative bioavailability (Frel) of itraconazole of greater than about 150% relative to the reference composition (e.g., commercially available SPORANOX®) under fed conditions, such as a relative bioavailability (Frel) of itraconazole of greater than about 160%, about 165%, about 170%, about 175%, or about 180%, such as about 180%, relative to the reference composition (e.g., commercially available SPORANOX®) under fed conditions.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

Ten exemplary compounds of Formula (I) were synthesized and tested for stability in blood.

### Example 1 Synthesis of Compound 1

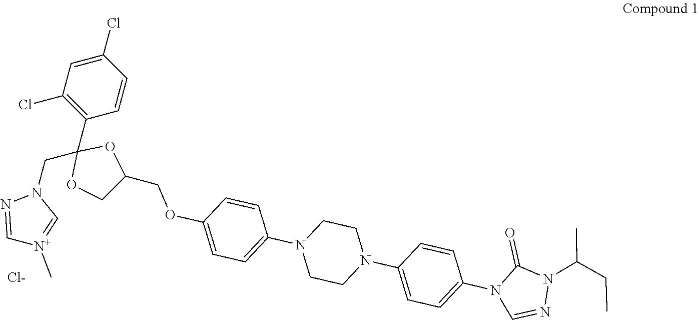 [SMILES: CCC(C)n1ncn(-c2ccc(N3CCN(c4ccc(OCC5COC(Cn6c[n+](C)cn6)(c6ccc(Cl)cc6Cl)O5)cc4)CC3)cc2)c1=O ; [Cl-]]

To a stirred solution of Itraconazole (1.50 g, 2.126 mmol, 1.00 eq.) in acetonitrile (20 mL) was added iodomethane (0.61 g, 4.298 mmol, 2.02 eq.). The reaction was heated at 70° C. for 16 h. LCMS indicated that the desired MS was formed. The reaction was cooled to room temperature, filtered and the filtrate was concentrated to give the crude product, which was purified by silica gel chromatography (eluent: dichloromethane/methanol=50/1-20/1) to give the iodide salt. The iodide salt was dissolved in 100 mL of acetonitrile, then 30 g of Cl⁻ ion exchange resin was added. The mixture was stirred at 15° C. for 16 h. ELSD and TLC indicated Cl⁻ and no I⁻. The mixture was filtered and the filtrate was concentrated to give a residue, which was purified again by flash chromatography (eluent: dichloromethane/methanol=50-20/1) to give 120 mg (7.5%) of Compound 1 (chloride salt) as a white solid.

LC-MS: (ESI, m/z): [M]⁺=719.40; RT=1.278 min

1H-NMR: (400 MHz, DMSO-d6, ppm): δ 10.20 (s, 1H), 9.13 (s, 1H), 8.34 (s, 1H), 7.76 (d, J=2.0 Hz, 1H), 7.67 (d, J=8.8 Hz, 1H), 7.56-7.50 (m, 3H), 7.11 (d, J=9.2 Hz, 2H), 6.99 (d, J=9.2 Hz, 2H), 6.84 (d, J=9.2 Hz, 2H), 5.05 (m, 2H), 4.42 (m, 1H), 4.11 (m, 1H), 3.94 (m, 2H), 3.82 (m, 1H), 3.78 (s, 3H), 3.71 (m, 1H), 3.33 (m, 4H), 3.19 (m, 4H), 1.69 (m, 2H), 1.30 (d, J=6.8 Hz, 3H), 0.80 (t, J=7.2 Hz, 3H).

### Example 2 Synthesis of Compound 2

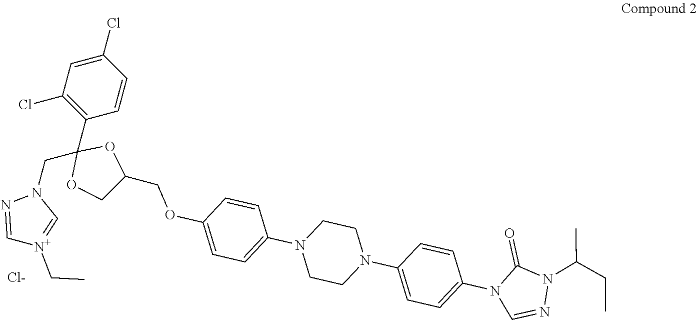 [SMILES: CCC(C)n1ncn(-c2ccc(N3CCN(c4ccc(OCC5COC(Cn6c[n+](CC)cn6)(c6ccc(Cl)cc6Cl)O5)cc4)CC3)cc2)c1=O ; [Cl-]]

To a stirred solution of Itraconazole (1.50 g, 2.126 mmol, 1.00 eq.) in acetonitrile (20 mL) was added iodoethane (0.66 g, 4.232 mmol, 1.99 eq.). The reaction was heated at 70° C. for 16 h. LCMS indicated that the desired MS was formed. The reaction was cooled to room temperature, filtered and the filtrate was concentrated to give the crude product, which was purified by silica gel chromatography (eluent: dichloromethane/methanol=50-20/1) to give the iodide salt. The iodide salt was dissolved in 100 mL of acetonitrile, then 30 g of Cl⁻ ion exchange resin was added. The mixture was stirred at 15° C. for 16 h. ELSD and TLC indicated Cl⁻ and no I⁻. The mixture was filtered and the filtrate was concentrated to give a residue, which was purified again by flash chromatography (eluent: dichloromethane/methanol=50-20/1) to give 150 mg (9.2%) of Compound 2 (chloride salt) as a white solid.

LC-MS: (ESI, m/z): [M]⁺=733.40; RT=1.385 min.

1H-NMR: (400 MHz, DMSO-d6, ppm): δ 10.28 (s, 1H), 9.24 (s, 1H), 8.34 (s, 1H), 7.76 (d, J=2.4 Hz, 1H), 7.67 (d, J=8.4 Hz, 1H), 7.56-7.50 (m, 3H), 7.12 (d, J=8.8 Hz, 2H), 6.98 (d, J=8.8 Hz, 2H), 6.83 (d, J=8.8 Hz, 2H), 5.04 (m, 2H), 4.42 (m, 1H), 4.19-4.15 (m, 3H), 3.94 (m, 2H), 3.82 (m, 1H), 3.70 (m, 1H), 3.33 (m, 4H), 3.19 (m, 4H), 1.69 (m, 2H), 1.37 (t, J=7.2 Hz, 3H), 1.30 (d, J=6.8 Hz, 3H), 0.81 (t, J=7.2 Hz, 3H).

### Example 3 Synthesis of Compound 3

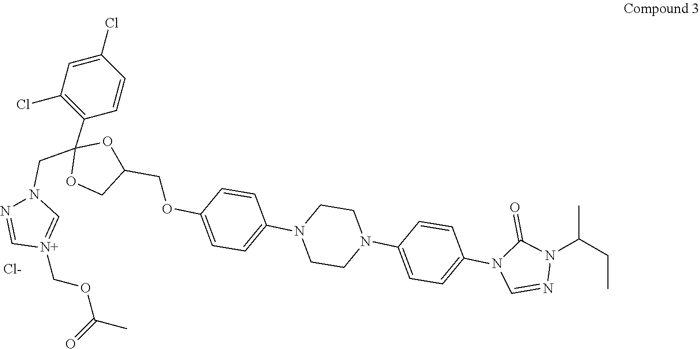 [SMILES: CCC(C)n1ncn(-c2ccc(N3CCN(c4ccc(OCC5COC(Cn6c[n+](COC(C)=O)cn6)(c6ccc(Cl)cc6Cl)O5)cc4)CC3)cc2)c1=O ; [Cl-]]

To a stirred solution of Itraconazole (1.0 g, 1.42 mmol, 1.0 eq.) in acetonitrile (15 mL) were added sodium iodide (425 mg, 2.83 mmol, 2.0 eq.) and chloromethyl acetate (308 mg, 2.83 mmol, 2.0 eq.). The reaction was heated at 90° C. for 2 h. LCMS indicated that most of Itraconazole was consumed and the desired MS was formed. The reaction was cooled to room temperature. The mixture was filtered and the filtrate was concentrated to give the crude product, which was purified by flash chromatography (eluent: dichloromethane/methanol=50-20/1) to give the iodide salt (300 mg). LCMS indicated that the purity was about 96%. The iodide salt was dissolved in 100 mL of acetonitrile, then 30 g of Cl⁻ ion exchange resin was added. The mixture was stirred at 15° C. for 16 h. LCMS indicated that the purity decreased to about 91%. ELSD indicated Cl⁻ and no I⁻. Then the mixture was filtered and the filtrate was concentrated to give the residue, which was lyophilized to give 140 mg (11.0%) of Compound 3 (chloride salt) as a light pink solid.

LC-MS: (ESI, m/z): [M]⁺=777.40; RT=1.272 min.

1H-NMR: (300 MHz, DMSO-d6, ppm): δ 10.46 (s, 1H), 9.38 (s, 1H), 8.34 (s, 1H), 7.76 (d, J=1.8 Hz, 1H), 7.65 (d, J=8.4 Hz, 1H), 7.56-7.50 (m, 3H), 7.11 (d, J=9.0 Hz, 2H), 6.99 (d, J=9.3 Hz, 2H), 6.84 (d, J=9.0 Hz, 2H), 6.10 (m, 2H), 5.10 (s, 2H), 4.40 (m, 1H), 4.12 (m, 1H), 3.94 (m, 2H), 3.78 (m, 2H), 3.32 (m, 4H, hidden in H₂O peak), 3.19 (m, 4H), 2.06 (s, 3H), 1.69 (m, 2H), 1.29 (d, J=6.6 Hz, 3H), 0.80 (t, J=7.2 Hz, 3H).

### Example 4 Synthesis of Compound 4

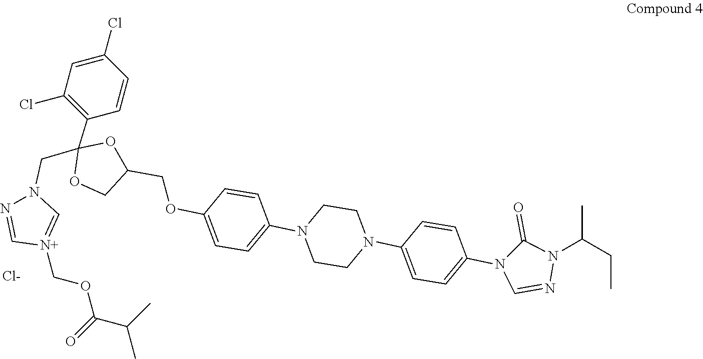 [SMILES: CCC(C)n1ncn(-c2ccc(N3CCN(c4ccc(OCC5COC(Cn6c[n+](COC(=O)C(C)C)cn6)(c6ccc(Cl)cc6Cl)O5)cc4)CC3)cc2)c1=O ; [Cl-]]

To a stirred solution of Itraconazole (1.0 g, 1.42 mmol, 1.0 eq.) in acetonitrile (15 mL) were added sodium iodide (425 mg, 2.83 mmol, 2.0 eq.) and chloromethyl 2-methylpropanoate (387 mg, 2.83 mmol, 2.0 eq). The reaction was heated at 90° C. for 2 h. LCMS indicated that most of Itraconazole was consumed and the desired MS was formed. The reaction was cooled to room temperature. The mixture was filtered and the filtrate was concentrated to give a residue, which was purified by silica gel chromatography (eluent: dichloromethane/methanol=50/1-20/1) to give 300 mg of a crude product. It was purified further by flash chromatography (eluent:dichloromethane/methanol=50-20/1) to give the iodide salt (220 mg). LCMS indicated that the purity was more than 95%. The iodide salt was dissolved in 100 mL of acetonitrile, then 30 g of Cl⁻ ion exchange resin was added. The mixture was stirred at 15° C. for 16 h. ELSD indicated Cl⁻ and no I⁻. The mixture was filtered and the filtrate was concentrated to give a residue, which was lyophilized to give 156.6 mg (12.1%) of Compound 4 (chloride salt) as a white solid.

LC-MS: (ESI, m/z): [M]⁺=805.55; RT=1.477 min.

1H-NMR: (300 MHz, DMSO-d6, ppm): δ 10.51 (s, 1H), 9.42 (s, 1H), 8.33 (s, 1H), 7.75 (d, J=2.1 Hz, 1H), 7.64 (d, J=8.4 Hz, 1H), 7.55-7.50 (m, 3H), 7.11 (d, J=9.0 Hz, 2H), 6.98 (d, J=9.0 Hz, 2H), 6.85 (d, J=9.0 Hz, 2H), 6.15 (s, 2H), 5.12 (s, 2H), 4.40 (m, 1H), 4.12 (m, 1H), 3.96 (m, 2H), 3.79 (m, 2H), 3.34 (m, 4H), 3.19 (m, 4H), 2.57 (m, 1H), 1.70 (m, 2H), 1.29 (d, J=6.6 Hz, 3H), 1.08 (dd, J1=6.9 Hz, J2=0.9 Hz, 6H), 0.83 (t, J=7.2 Hz, 3H).

### Example 5 Synthesis of Compound 5

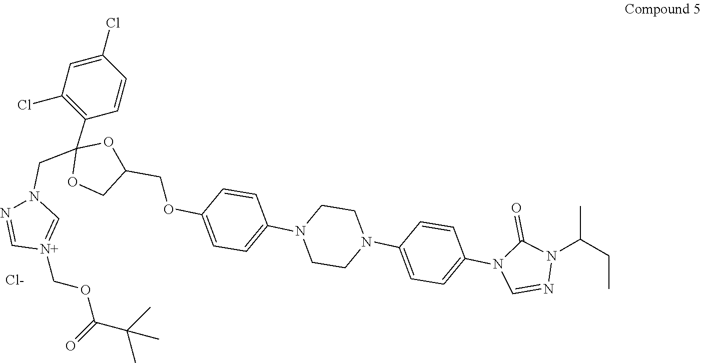 [SMILES: CCC(C)n1ncn(-c2ccc(N3CCN(c4ccc(OCC5COC(Cn6c[n+](COC(=O)C(C)(C)C)cn6)(c6ccc(Cl)cc6Cl)O5)cc4)CC3)cc2)c1=O ; [Cl-]]

To a stirred solution of Itraconazole (706 mg, 1.0 mmol, 1.0 eq) in acetonitrile (20 mL) were added sodium iodide (300 mg, 2.0 mmol, 2.0 eq) and chloromethyl 2,2-dimethylpropanoate (301 mg, 2.0 mmol, 2.0 eq). The reaction was heated at 90° C. for 2 h. LCMS indicated that most of Itraconazole was consumed and about 80% of the desired compound was formed. The reaction mixture was filtered and the filtrate was concentrated to give a residue, which was purified by prep-HPLC (ACN/H₂O/0.1% HCOOH). To the preparation solution was added excess sodium chloride. The organic phase was separated, dried over Na₂SO₄, filtered and concentrated to give 200 mg of crude product as formic acid salt. The salt was dissolved in 100 mL of acetonitrile, then 30 g of Cl⁻ ion exchange resin was added. The mixture was stirred for 16 h at 15° C. ELSD indicated Cl⁻. The mixture was filtered, concentrated to give a residue, which was lyophilized to give 111.8 mg (12.8%) of Compound 5 (chloride salt) as a white solid. NMR indicated no HCOO—.

LC-MS: (ESI, m/z): [M]⁺=819.40; RT=2.213 min.

1H-NMR: (300 MHz, DMSO-d6, ppm): δ 10.52 (s, 1H), 9.43 (s, 1H), 8.35 (s, 1H), 7.76 (d, J=2.1 Hz, 1H), 7.63 (d, J=8.4 Hz, 1H), 7.55-7.50 (m, 3H), 7.11 (d, J=9.0 Hz, 2H), 6.98 (d, J=9.0 Hz, 2H), 6.85 (d, J=9.3 Hz, 2H), 6.16 (s, 2H), 5.13 (s, 2H), 4.39 (m, 1H), 4.12 (m, 1H), 3.96 (m, 2H), 3.78 (m, 2H), 3.34 (m, 4H, hidden in H₂O peak), 3.19 (m, 4H), 1.69 (m, 2H), 1.29 (d, J=6.9 Hz, 3H), 1.14 (s, 9H), 0.80 (t, J=7.2 Hz, 3H).

### Example 6 Synthesis of Compound 6

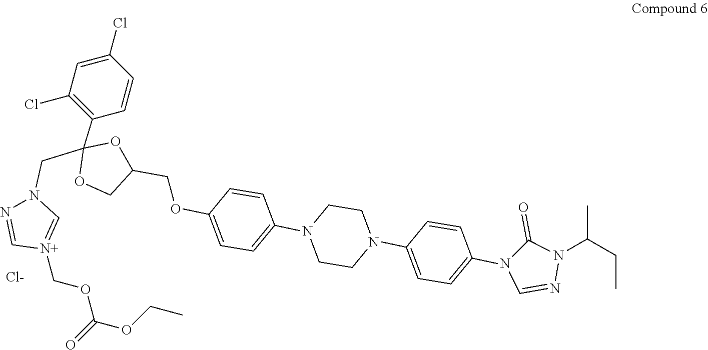 [SMILES: CCOC(=O)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]]

To a stirred solution of Itraconazole (1.0 g, 1.42 mmol, 1.0 eq.) in acetonitrile (20 mL) were added sodium iodide (318.6 mg, 2.13 mmol, 1.5 eq.) and chloromethyl ethyl carbonate (393 mg, 2.83 mmol, 2.0 eq.). The reaction was heated at 90° C. for 2 h. LCMS indicated that most of Itraconazole was consumed and the desired MS was formed. The reaction was cooled to room temperature, filtered and the filtrate was concentrated to give a residue, which was purified by silica gel chromatography (eluent: dichloromethane/methanol=50-20/1) to give 200 mg of the iodide salt. LCMS indicated that the purity was more than 90%. 200 mg of the iodide salt was dissolved in 100 mL of acetonitrile, then 30 g of Cl⁻ ion exchange resin was added. The mixture was stirred at 15° C. for 16 h. ELSD indicated Cl⁻ and no I⁻. The mixture was filtered and the filtrate was concentrated to give a residue, which was lyophilized to give 105.7 mg (8.4%) of Compound 6 (chloride salt) as a white solid.

LC-MS: (ESI, m/z): [M]⁺=807.40; RT=1.313 min.

1H-NMR: (400 MHz, DMSO-d6, ppm): δ 10.51 (s, 1H), 9.42 (s, 1H), 8.34 (s, 1H), 7.76 (d, J=2.0 Hz, 1H), 7.65 (d, J=8.4 Hz, 1H), 7.55-7.50 (m, 3H), 7.11 (d, J=9.2 Hz, 2H), 6.98 (d, J=9.2 Hz, 2H), 6.84 (d, J=9.2 Hz, 2H), 6.15 (m, 2H), 5.11 (s, 2H), 4.39 (m, 1H), 4.15 (m, 1H), 3.95 (m, 2H), 3.79 (m, 2H), 3.34 (m, 4H), 3.19 (m, 4H), 1.70 (m, 2H), 1.29 (d, J=6.8 Hz, 3H), 1.21 (t, J=7.2 Hz, 3H), 0.80 (t, J=7.2 Hz, 3H).

### Example 7 Synthesis of Compound 7

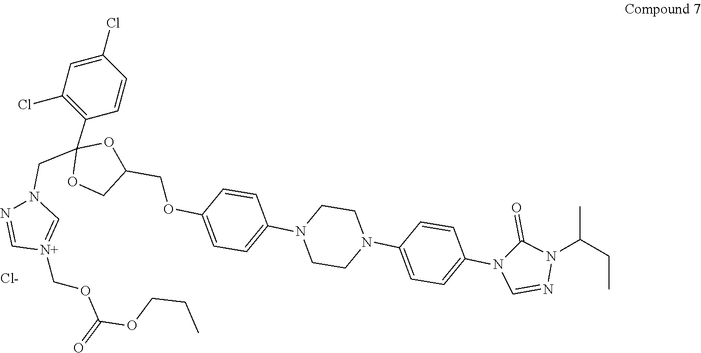 [SMILES: CCCOC(=O)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]]

To a stirred solution of Itraconazole (1.0 g, 1.42 mmol, 1.0 eq.) in acetonitrile (20 mL) were added sodium iodide (318.6 mg, 2.13 mmol, 1.5 eq.) and chloromethyl propyl carbonate (432 mg, 2.83 mmol, 2.0 eq.). The reaction was heated at 90° C. for 2 h. LCMS indicated that most of Itraconazole was consumed and the desired MS was formed. The reaction was cooled to room temperature, filtered and the filtrate was concentrated to give a residue, which was purified by silica gel chromatography (eluent: dichloromethane/methanol=50-20/1) to give 200 mg of the iodide salt. LCMS indicated that the purity was more than 90%. The iodide salt was dissolved in 100 mL of acetonitrile, then 30 g of Cl⁻ ion exchange resin was added. The mixture was stirred at 15° C. for 16 h. ELSD indicated Cl⁻ and no I⁻. The mixture was filtered and the filtrate was concentrated to give a residue, which was lyophilized to give 104.1 mg (8.4%) of Compound 7 (Cl⁻ salt) as a white solid.

LC-MS: (ESI, m/z): [M]⁺=812.50; RT=1.367 min.

1H-NMR: (400 MHz, DMSO-d6, ppm): δ 10.52 (s, 1H), 9.42 (s, 1H), 8.34 (s, 1H), 7.76 (d, J=2.0 Hz, 1H), 7.65 (d, J=8.4 Hz, 1H), 7.55-7.50 (m, 3H), 7.11 (d, J=9.2 Hz, 2H), 6.98 (d, J=9.2 Hz, 2H), 6.84 (d, J=9.2 Hz, 2H), 6.15 (m, 2H), 5.11 (s, 2H), 4.39 (m, 1H), 4.15-4.06 (m, 3H), 3.95 (m, 2H), 3.78 (m, 2H), 3.32 (m, 4H), 3.19 (m, 4H), 1.72-1.58 (m, 4H), 1.29 (d, J=6.8 Hz, 3H), 1.21 (t, J=7.2 Hz, 3H), 0.87 (t, J=7.2 Hz, 3H), 0.80 (t, J=7.2 Hz, 3H).

### Example 8 Synthesis of Compound 8

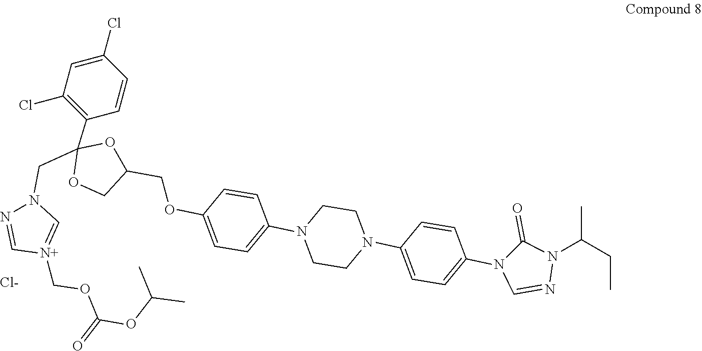 [SMILES: CCC(C)n1ncn(-c2ccc(N3CCN(c4ccc(OCC5COC(Cn6c[n+](COC(=O)OC(C)C)cn6)(c6ccc(Cl)cc6Cl)O5)cc4)CC3)cc2)c1=O ; [Cl-]]

To a stirred solution of Itraconazole (1.0 g, 1.42 mmol, 1.0 eq.) in acetonitrile (20 mL) were added sodium iodide (425 mg, 2.83 mmol, 2.0 eq.) and chloromethyl propan-2-yl carbonate (432 mg, 2.83 mmol, 2.0 eq.). The reaction was heated at 90° C. for 2 h. LCMS indicated that most of Itraconazole was consumed and the desired MS was formed. The reaction was cooled to room temperature, filtered and the filtrate was concentrated to give a residue, which was purified by silica gel chromatography (eluent: dichloromethane/methanol=50-20/1) to give 200 mg of the iodide salt. LCMS indicated that the purity was more than 90%. The iodide salt was dissolved in 100 mL of acetonitrile, then 30 g of Cl⁻ ion exchange resin was added. The mixture was stirred at 15° C. for 16 h. ELSD indicated Cl⁻ and no I⁻. The mixture was filtered and the filtrate was concentrated to give a residue, which was lyophilized to give 127.1 mg (10.3%) of Compound 8 (chloride salt) as a white solid.

LC-MS: (ESI, m/z): [M]⁺=821.40; RT=1.586 min.

1H-NMR: (300 MHz, DMSO-d6, ppm): δ 10.50 (s, 1H), 9.42 (s, 1H), 8.35 (s, 1H), 7.76 (d, J=1.8 Hz, 1H), 7.67 (d, J=8.7 Hz, 1H), 7.55-7.50 (m, 3H), 7.11 (d, J=9.3 Hz, 2H), 6.98 (d, J=9.0 Hz, 2H), 6.84 (d, J=9.3 Hz, 2H), 6.15 (s, 2H), 5.11 (s, 2H), 4.80 (m, 1H), 4.40 (m, 1H), 4.12 (m, 1H), 3.96 (m, 2H), 3.78 (m, 2H), 3.34 (m, 4H), 3.19 (m, 4H), 1.68 (m, 2H), 1.29 (d, J=6.6 Hz, 3H), 1.25 (dd, J1=6.3 Hz, J2=2.1 Hz, 6H), 0.80 (t, J=7.2 Hz, 3H).

### Example 9 Synthesis of Compound 9

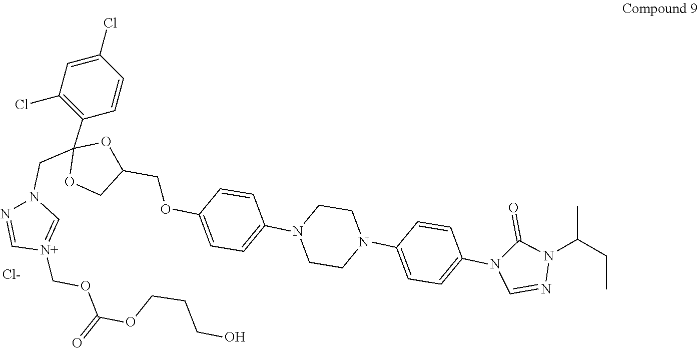 [SMILES: CCC(C)n1ncn(-c2ccc(N3CCN(c4ccc(OCC5COC(Cn6c[n+](COC(=O)OCCCO)cn6)(c6ccc(Cl)cc6Cl)O5)cc4)CC3)cc2)c1=O ; [Cl-]]

To a stirred solution of Itraconazole (2.15 g, 3.05 mmol, 1.0 eq.) in acetonitrile (50 mL) were added sodium iodide (0.69 g, 4.60 mmol, 1.5 eq.) and chloromethyl 3-hydroxypropanyl carbonate (1.03 g, 6.09 mmol, 2.0 eq.). The reaction was heated at 90° C. for 2 h. LCMS indicated that most of Itraconazole was consumed and the desired MS was formed. The reaction was cooled to room temperature, filtered and the filtrate was concentrated to give a residue, which was purified by silica gel chromatography (eluent: dichloromethane/methanol=50-20/1) to give 200 mg of the iodide salt. LCMS indicated that the purity was more than 90%. The iodide salt was dissolved in 100 mL of ACN, then 30 g of Cl⁻ ion exchange resin was added. The mixture was stirred at 15° C. for 16 h. ELSD indicated Cl⁻ and no I⁻. The mixture was filtered and the filtrate was concentrated to give a residue, which was purified by silica gel chromatography again (eluent: dichloromethane/methanol=50-20/1) and then lyophilized to give 100.4 mg (3.5%) of Compound 9 (chloride salt) as a white solid.

LC-MS: (ESI, m/z): [M]⁺=837.40; RT=4.194 min.

1H-NMR: (400 MHz, DMSO-d6, ppm): δ 10.51 (s, 1H), 9.41 (s, 1H), 8.34 (s, 1H), 7.75 (d, J=2.0 Hz, 1H), 7.65 (d, J=8.4 Hz, 1H), 7.55-7.50 (m, 3H), 7.11 (d, J=8.8 Hz, 2H), 6.98 (d, J=8.8 Hz, 2H), 6.84 (d, J=9.2 Hz, 2H), 6.15 (m, 2H), 5.11 (s, 2H), 4.61 (brs, 1H), 4.40 (m, 1H), 4.21 (t, J=6.4 Hz, 2H), 4.12 (m, 1H), 3.96 (m, 2H), 3.79 (m, 2H), 3.44 (m, 2H), 3.33 (m, 4H), 3.19 (m, 4H), 1.77-1.64 (m, 4H), 1.28 (m, 3H), 0.80 (t, J=7.2 Hz, 3H).

Chloromethyl 3-hydroxypropanyl carbonate as used above was synthesized as follows.

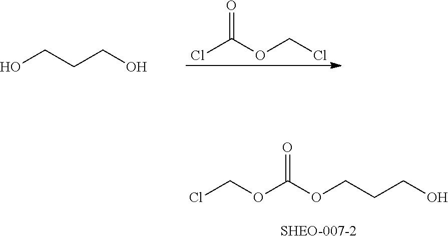 [SMILES: OCCCO ; O=C(Cl)OCCl ; O=C(OCCl)OCCCO]

Under N₂ atmosphere, to a stirred solution of propane-1,3-diol (3.8 g, 49.94 mmol, 1.0 eq.) in DCM (50 mL) was added TEA (12.63 g, 124.82 mmol, 2.50 eq.). Then chloromethyl carbonochloridate (6.44 g, 49.94 mmol, 1.00 eq.) was added slowly at 5° C. The reaction system was stirred at this temperature for 3 h. TLC indicated that a new spot was formed. Then 50 mL of saturated NaHCO₃ solution and 50 mL of DCM were added. The organic phase was separated, dried over Na₂SO₄, filtered and concentrated to give 3.3 g crude chloromethyl 3-hydroxypropanyl carbonate as a yellow oil (39.20%).

### Example 10 Synthesis of Compound 10

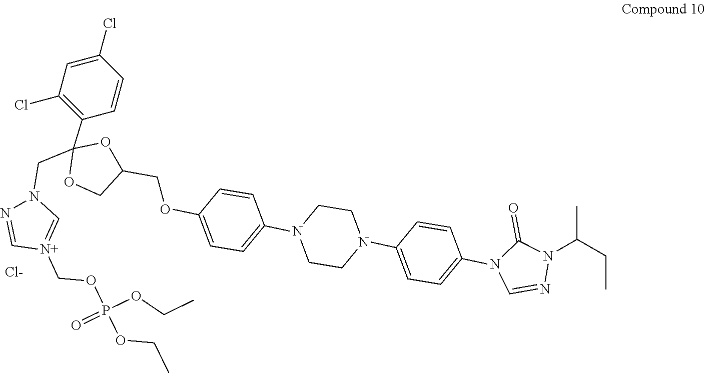 [SMILES: CCOP(=O)(OCC)OC[n+]1cnn(CC2(c3ccc(Cl)cc3Cl)OCC(COc3ccc(N4CCN(c5ccc(-n6cnn(C(C)CC)c6=O)cc5)CC4)cc3)O2)c1 ; [Cl-]]

To a stirred solution of Itraconazole (580 mg, 0.82 mmol, 1 eq.) in acetonitrile (15 mL) were added sodium iodide (184.8 mg, 1.23 mmol, 1.5 eq.) and chloromethyl diethyl phosphate (333.00 mg, 1.644 mmol, 2.0 eq.). The reaction was heated at 90° C. for 2 h. LCMS indicated that most of Itraconazole was consumed and the desired MS was formed. The reaction was cooled to room temperature, filtered and the filtrate was concentrated to give a residue, which was purified by silica gel chromatography (eluent: dichloromethane/methanol=50-20/1) to give of the iodide salt (280 mg), and LCMS indicated that the purity was more than 90%. The iodide salt (200 mg) of was dissolved in 100 mL of ACN, then 30 g of Cl⁻ ion exchange resin was added. The mixture was stirred at 15° C. for 16 h. ELSD and TLC indicated Cl⁻ and no I⁻. The mixture was filtered and the filtrate was concentrated to give a residue, which was lyophilized to give 107.5 mg (13.5%) of Compound 10 (chloride salt) as a white solid.

LC-MS: (ESI, m/z): [M]⁺=873.05; RT=3.848 min.

1H-NMR: (300 MHz, DMSO-d6, ppm): δ 10.58 (s, 1H), 9.49 (s, 1H), 8.34 (s, 1H), 7.76 (d, J=1.6 Hz, 1H), 7.64 (d, J=8.4 Hz, 1H), 7.55-7.50 (m, 3H), 7.11 (d, J=9.2 Hz, 2H), 6.98 (d, J=8.8 Hz, 2H), 6.85 (d, J=8.8 Hz, 2H), 6.10 (m, 2H), 5.16 (s, 2H), 4.40 (m, 1H), 4.15-4.05 (m, 5H), 3.95 (m, 2H), 3.79 (m, 2H), 3.33 (m, 4H), 3.19 (m, 4H), 1.69 (m, 2H), 1.30-1.23 (m, 9H), 0.80 (t, J=7.2 Hz, 3H).

Chloromethyl diethyl phosphate was synthesized as follows.

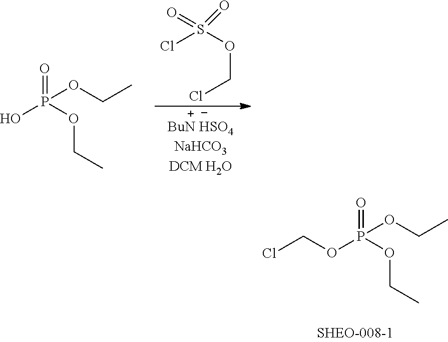

To a solution of diethoxyphosphinic acid (5.0 g, 32.45 mmol, 1.0 eq.) in H₂O (200 mL) were added NaHCO₃ (10.90 g, 129.784 mmol, 4.00 equiv), Bu₄NHSO₄ (1.10 g, 3.245 mmol, 0.1 eq.) and DCM (20 mL). Then chloromethyl sulfurochloridate (6.42 g, 38.91 mmol, 1.20 eq.) was added slowly at 0° C. The reaction system was stirred at 15° C. for 16 h. The organic phase was separated, dried over Na₂SO₄, filtered and concentrated to give the residue, which was purified by silica gel chromatography (eluent: PE/EA-5/1) to give chloromethyl diethyl phosphate (335 mg, 5.10%).

LC-MS: (ESI, m/z): [M]⁺=203.10

### Example 10 Compound Stability Test in Blood

### Preparation of Stock Solutions

One (1) mM test compound working solution was prepared in DMSO. One (1) mM propantheline working solution was prepared in acetonitrile, wherein propantheline was used as positive control in this assay.

Ten (10) μL of each test compound solution and the control compound solution were respectively added into a 96 deep well plate, and 40 μL of acetonitrile:water (V/V=1:1) was added to each well to dilute the 1 mM test compound solution to 200 μM working solution.

Mouse blood (male CD-1 mouse blood, Pharmaron, Lot# PH-ADME-Mouse-20180329) or human blood (Pharmaron, Lot# PH-ADME-Human-20180329) of 398 μL was added to each well of an incubation plate, and then the incubation plate was pre-warmed at 37° C. for 15 minutes. After the pre-incubation, 2 μL of 200 μM working solution prepared above was spiked to the pre-warmed 398 μL blood to reach a final concentration of 1 μM. The final concentration of DMSO was 0.1% and the final concentration of acetonitrile was 0.2%. The assay was performed in duplicate.

The incubation plate was incubated at 37° C. water bath with shaking at 50 rpm, and aliquots of 50 μL were taken from each well of the incubation plate at 0, 10, 30, 60, 90, 120 and 180 minutes.

The reaction was stopped by the addition of 50 μL of pure water. The plate was vortexed for 30 seconds at 1,000 rpm and then added with 300 μL of cold acetonitrile containing internal standards (100 nM aprozolam, 200 nM caffeine, 100 nM tolbutamide).

All samples were vortexed for 10 minutes, followed by centrifugation at 14,000 rpm for 15 minutes to precipitate proteins. 100 μL of each supernatant was transferred to a new plate, and diluted with ultrapure water according to the LC-MS signal response and peak shape.

Samples were analyzed by LC-MS/MS.

### Chromatographic Condition:

LC system: ACQUITY UPLC® I-Class
MS analysis: Waters XEVO® TQ-D with an ESI interface
Column temperature: 40° C.
Injection volume: 3 μL
Column: Waters XSelect HSS T3 C18, 2.5 μm, 2.1×50 mm column
Mobile phase: 0.1% formic acid in water (A) and 0.1% formic acid in acetonitrile (B)

### MS Parameters:

Ion source: Turbo spray
Ionization model: ESI
Scan type: MRM
Cone gas: 150 L/h
Desolvation gas: 1000 L/h
Desolvation temperature: 500° C.
Capillary voltage: 3.00 KV

All calculations were carried out using Microsoft Excel. Peak area ratios were determined from extracted ion chromatograms, and percent compounds remaining at each time point were calculated by the following equation:

Remaining Percentage_{t min}(%)=Peak Area Ratio_{t min}/Peak Area Ratio_{0 min}×100, where Peak Area Ratio_{t min} referred to peak area ratio of control and test compounds at t min; and Peak Area Ratio_{0 min} referred to peak area ratio of control and test compounds at zero time point. The in vitro half-life (in vitro t_{1/2}) was determined from the slope value: In vitro T_{1/2}=−(0.693/k).

The results were summarized in Table 1 and 2 below. All exemplary compounds of Formula (I) were suitable for use as a prodrug of itraconazole. Compound 3 was more stable among exemplary compounds in mouse and human blood. Stability of a prodrug compound in blood is of importance for the compound to be efficacious in e.g., preclinical studies, and/or in clinical studies and/or in a regulatory agency approved drug product.

TABLE 1
The stability of exemplary compounds in mouse blood

| | Remaining Percentages (%) | | | | | | | T_{1/2} |
|---|---|---|---|---|---|---|---|---|
| | 0 min | 10 min | 30 min | 60 min | 90 min | 120 min | 180 min | (min) |
| Propantheline | 100 | 88.8 | 62.4 | 34.8 | 20.4 | 12.3 | 4.44 | 38.8 |
| Compound 1 | 100 | 2.01 | 0.00 | 0.00 | 0.00 | 0.00 | 0.00 | 1.77 |
| Compound 2 | 100 | 40.1 | 16.2 | 3.79 | 0.90 | 0.175 | 0.00 | 11.8 |
| Compound 3 | 100 | 88.1 | 67.1 | 46.3 | 30.3 | 24.4 | 12.0 | 59.0 |
| Compound 4 | 100 | 31.5 | 4.04 | BLOD | BLOD | BLOD | BLOD | 6.03 |
| Compound 5 | 100 | 23.9 | 1.48 | BLOD | BLOD | BLOD | BLOD | 4.84 |
| Compound 6 | 100 | 51.5 | 12.6 | 3.10 | 0.595 | 0.186 | 0.232 | 10.0 |
| Compound 7 | 100 | 37.3 | BLOD | BLOD | BLOD | BLOD | BLOD | 7.12 |
| Compound 8 | 100 | 71.2 | 40.2 | 14.8 | 7.0 | 3.99 | 1.03 | 21.93 |

BLOD: below detection limitattion

TABLE 2
The stability of exemplary compounds in human blood

| | Remaining Percentages (%) | | | | | | | T_{1/2} |
|---|---|---|---|---|---|---|---|---|
| | 0 min | 10 min | 30 min | 60 min | 90 min | 120 min | 180 min | (min) |
| Propantheline | 100 | 85.5 | 55.8 | 29.2 | 15.2 | 6.83 | 1.84 | 32.8 |
| Compound 1 | 100 | 68.5 | 32.6 | 12.3 | 7.80 | 2.02 | 0.36 | 23.1 |
| Compound 2 | 100 | 60.3 | 26.1 | 8.47 | 3.59 | 1.66 | 0.30 | 15.6 |
| Compound 3 | 100 | 94.0 | 82.3 | 67.5 | 61.5 | 55.3 | 34.3 | 123 |
| Compound 4 | 100 | 61.0 | 30.2 | 8.93 | 4.67 | BLOD | BLOD | 17.7 |
| Compound 5 | 100 | 69.4 | 30.4 | 9.76 | 5.50 | 2.08 | BLOD | 18.1 |
| Compound 6 | 100 | 94.2 | 77.3 | 55.3 | 41.1 | 34.0 | 15.2 | 67.4 |
| Compound 7 | 100 | 25.3 | BLOD | BLOD | BLOD | BLOD | BLOD | 5.07 |
| Compound 8 | 100 | 31.6 | 1.40 | 0.785 | 0.616 | BLOD | BLOD | 6.03 |

BLOD: below limitation of detection

### Example 11 Itraconazole Release in Blood

In the blood stability assay described above, the release of itraconazole coincide with and as a result of metabolism of the itraconazole prodrugs was also monitored by LC-MS/MS.

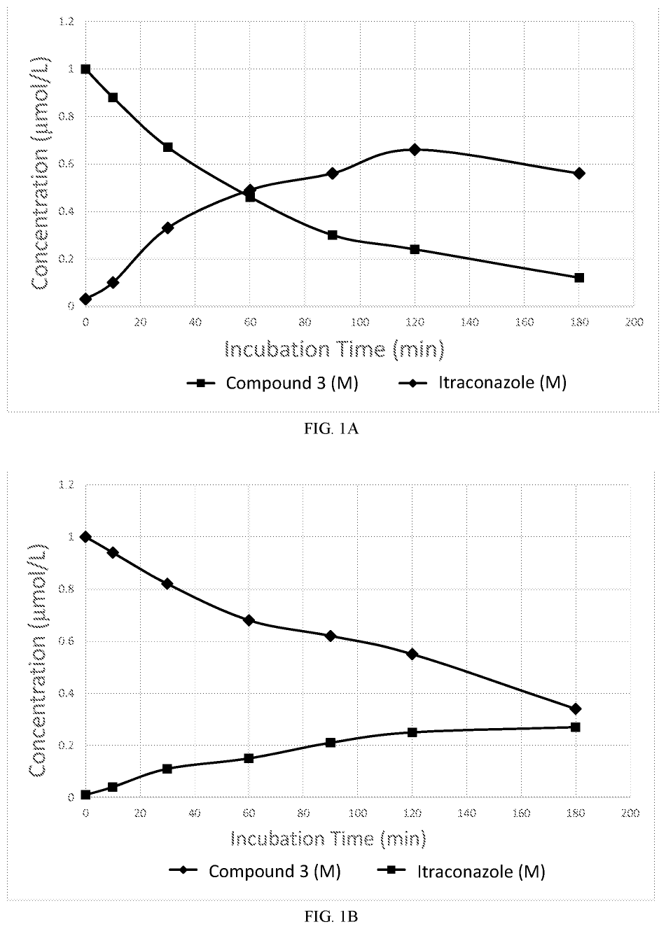

The results of compound 3 were shown in Table 3 and FIGS. 1A-1B.

TABLE 3
Concentrations of Compound 3 and released itraconazole in
mouse and human blood

| | | Concentration (mol/L) | | | | | | |
|---|---|---|---|---|---|---|---|---|
| | | 0 min | 10 min | 30 min | 60 min | 90 min | 120 min | 180 min |
| Mouse | Compound 3 | 1.00 | 0.88 | 0.67 | 0.46 | 0.30 | 0.24 | 0.12 |
| Blood | Itraconazole | 0.00 | 0.10 | 0.33 | 0.49 | 0.56 | 0.66 | 0.56 |
| Human | Compound 3 | 1.00 | 0.94 | 0.82 | 0.68 | 0.62 | 0.55 | 0.34 |
| Blood | Itraconazole | 0.00 | 0.04 | 0.11 | 0.15 | 0.21 | 0.25 | 0.27 |

The data demonstrated that once the prodrug compound of the disclosure getting into the blood stream of either mice or human, itraconazole was slowly released while the compound was metabolized.

### Example 12 Pharmacokinetic Study of Itraconazole Release in Mice after Intravenous and Oral Dosing of Compound 3

Compound 3 was dissolved in DMSO:PEG400:water (1:4:5, v/v/v) with a final concentration of 0.2 mg/ml or 0.5 mg/ml. The 0.2 mg/ml solution was used for intravenous dosing, and the 0.5 mg/ml solution was for oral dosing.

Three male C57BL/6 mice were intravenously administered with Compound 3 at a dose of 1.0 mg/kg, and blood samples were collected at 2 min, 15 min, 30 min, 1 h, 2 h, 4 h, 6 h, 8 h, and 24 h post administration.

Three male C57BL/6 mice were orally given Compound 3 at a dose of 5.0 mg/kg, and blood samples were collected at 15 min, 30 min, 1 h, 2 h, 4 h, 6 h, 8 h, and 24 h post administration.

No mice were observed with abnormal clinical symptoms during the test. And dose levels were corrected in PK parameter calculation due to the salt factor.

The blood samples were subject to LC-MS/MS analysis, to monitor the plasma concentrations of both the parent compound and Itraconazole.

### HPLC:

Instrument: Prominence (Degasser DGU-20A5R(C), Serial NO. L20705310737 IX; Liquid Chromatograph LC-30AD Serial NO. L20555306326 AE and L20555306328 AE; Communications Bus Module CBM-20A, Serial NO. L20235326202 CD, Auto Sampler SIL-30AC, Serial NO.L20565303024 AE; Rack changer II: L20585300741 SS

Column: Waters XSELECT HSS T3 2.5 μm 2.1×50 mm

Mobile phase: 5% Acetonitrile in Water (0.1% Formic acid) (A) and 95% Acetonitrile in Water (0.1% Formic acid) (B)

Gradient: flow rate at 0.6 mL/min

| Time (min) | A (%) | B (%) |
|---|---|---|
| 0.30 | 95.0 | 5.00 |
| 0.90 | 45.0 | 55.0 |
| 2.30 | 0.00 | 100 |
| 2.50 | 0.00 | 100 |
| 2.51 | 95.0 | 5.00 |

Injection volumn: 10 μL

### MS:

AB Sciex Triple Quad 5500 LC/MS/MS instrument (Serial NO. BB212611510)

### Quantification: Internal Standard Method

The plasma itraconazole concentrations and pharmacokinetic parameters after i.v. dosing of Compound 3 were summarized in Table 4 and 5.

TABLE 4
Plasma itroconazole concentrations after intravenous
dosing of Compound 3
IV Dose 0.860 mg/kg

| Time | Concentration (ng/mL) | | | Mean | SD | CV |
|---|---|---|---|---|---|---|
| (h) | Mouse 1 | Mouse 2 | Mouse 3 | (ng/mL) | (ng/mL) | (%) |
| 0.033 | 3850 | 3540 | 3840 | 3743 | 176 | 4.71 |
| 0.25 | 771 | 786 | 894 | 817 | 67 | 8.21 |
| 0.5 | 555 | 465 | 453 | 491 | 56 | 11.4 |
| 1 | 246 | 244 | 308 | 266 | 36 | 13.7 |
| 2 | 155 | 130 | 167 | 151 | 19 | 12.5 |
| 4 | 60.9 | 39.9 | 50.9 | 50.6 | 10.5 | 20.8 |
| 6 | 26.0 | 16.2 | 22.1 | 21.4 | 4.9 | 23.0 |
| 8 | 10.4 | BLOQ | 7.82 | 9.1 | NA | NA |
| 24 | BLOQ | BLOQ | BLOQ | NA | NA | NA |

BLOQ: below limitation of quatification

TABLE 5
Itraconazole pharmacokinetic characteristics after intravenous
dosing of Compound 3

| PK parameters | Unit | Mouse 1 | Mouse 2 | Mouse 3 | Mean | SD | CV(%) |
|---|---|---|---|---|---|---|---|
| Cl_obs | mL/min/kg | 9.10 | 10.4 | 9.02 | 9.5 | 0.8 | 8.13 |
| T_{1/2} | h | 1.57 | 1.33 | 1.48 | 1.46 | 0.12 | 8.21 |
| C₀ | ng/mL | 4917 | 4450 | 4793 | 4720 | 242 | 5.12 |
| AUCₗₐₛₜ | h*ng/mL | 1552 | 1348 | 1573 | 1491 | 124 | 8.34 |
| AUC_{Inf} | h*ng/mL | 1575 | 1379 | 1590 | 1515 | 118 | 7.77 |
| AUC_% Extrap_obs | % | 1.49 | 2.26 | 1.05 | 1.60 | 0.61 | 38.1 |
| MRT_{Inf}_obs | h | 1.23 | 1.00 | 1.13 | 1.12 | 0.11 | 10.2 |
| AUCₗₐₛₜ/D | h*mg/mL | 1805 | 1568 | 1839 | 1734 | 145 | 8.34 |
| Vₛₛ_obs | L/kg | 0.672 | 0.626 | 0.629 | 0.635 | 0.033 | 5.13 |

The plasma itraconazole concentrations and pharmacokinetic parameters after oral dosing of Compound 3 were summarized in Table 6 and 7.

TABLE 6
Plasma itroconazole concentrations after oral dosing
of Compound 3
PO Dose 4.30 mg/kg

| Time | Concentration (ng/mL) | | | Mean | SD | CV |
|---|---|---|---|---|---|---|
| (h) | Mouse 4 | Mouse 5 | Mouse 6 | (ng/mL) | (ng/mL) | (%) |
| 0.25 | 16.1 | 42.1 | 13.9 | 24.0 | 15.7 | 65.3 |
| 0.5 | 66.9 | 161 | 61.8 | 97 | 56 | 57.8 |
| 1 | 228 | 389 | 218 | 278 | 96 | 34.5 |
| 2 | 204 | 262 | 200 | 222 | 35 | 15.6 |
| 4 | 85.6 | 124 | 78.3 | 96 | 25 | 25.6 |
| 6 | 55.2 | 66.9 | 42.5 | 54.9 | 12.2 | 22.2 |
| 8 | 37.3 | 53.7 | 34.2 | 41.7 | 10.5 | 25.1 |
| 24 | BLOQ | BLOQ | BLOQ | NA | NA | NA |

BLOQ: below limitation of quatification

TABLE 7
Itraconazole pharmacokinetic characteristics after oral dosing
of Compound 3

| PK parameters | Unit | Mouse 4 | Mouse 5 | Mouse 6 | Mean | SD | CV (%) |
|---|---|---|---|---|---|---|---|
| T_{1/2} | h | 3.34 | 3.31 | 3.35 | 3.33 | 0.02 | 0.53 |
| Tₘₐₓ | h | 1 | 1 | 1 | 1.00 | 0.00 | 0.00 |
| Cₘₐₓ | ng/mL | 228 | 389 | 218 | 278 | 96 | 34.5 |
| AUCₗₐₛₜ | h*ng/mL | 825 | 1191 | 766 | 927 | 230 | 24.8 |
| AUC_{Inf} | h*ng/mL | 1005 | 1448 | 931 | 1128 | 280 | 24.8 |
| AUC_% Extrap_obs | % | 17.9 | 17.7 | 17.7 | 17.8 | 0.1 | 0.47 |
| MRT_{Inf}_obs | h | 4.72 | 4.57 | 4.62 | 4.64 | 0.08 | 1.67 |
| AUCₗₐₛₜ/D | h*mg/mL | 192 | 277 | 178 | 216 | 54 | 24.8 |
| F | % | 13.3 | 19.1 | 12.3 | 14.9 | 3.7 | 24.8 |

The results of mouse PK study on Compound 3 showed that after intravenous dosing of 0.860 mpk (milligram per kilogram body weight), Itraconazole was released with an average AUC (area under the curve) of about 1500 h*ng/mL, and with an average T_{1/2} of 1.5 hour.

The results of mouse PK study on Compound 3 showed that after oral dosing of 4.30 mpk, Itraconazole was released with an average AUC of about 1000 h*ng/mL, and with an average T_{1/2} of 3.3 hour. Cmax was reached at about 1 hour after dosing and was at the level of about 300 ng/mL.

### Example 13 Pharmacokinetic Study of Itraconazole Release in Mice after Intravenous and Oral Dosing of Compound 6

Compound 6 was dissolved in DMSO:PEG400:water (1:4:5, v/v/v) with a final concentration of 0.2 mg/ml or 0.5 mg/ml. The 0.2 mg/ml solution was used for intravenous dosing, and the 0.5 mg/ml solution was for oral dosing.

Three male C57BL/6 mice were intravenously administered with Compound 6 at a dose of 1.0 mg/kg, and blood samples were collected at 2 min, 15 min, 30 min, 1 h, 2 h, 4 h, 6 h, 8 h, and 24 h post administration.

Three male C57BL/6 mice were orally given Compound 6 at a dose of 5.0 mg/kg, and blood samples were collected at 15 min, 30 min, 1 h, 2 h, 4 h, 6 h, 8 h, and 24 h post administration.

No mice were observed with abnormal clinical symptoms during the test. And dose levels were corrected in PK parameter calculation due to the salt factor.

The blood samples were subject to LC-MS/MS analysis, to monitor the plasma concentrations of both the parent compound and Itraconazole.

### HPLC:

Instrument: Prominence (Degasser DGU-20A5R(C), Serial NO. L20705310737 IX; Liquid Chromatograph LC-30AD Serial NO. L20555306326 AE and L20555306328 AE; Communications Bus Module CBM-20A, Serial NO. L20235326202 CD, Auto Sampler SIL-30AC, Serial NO.L20565303024 AE; Rack changer II: L20585300741 SS

Column: Waters XSELECT HSS T3 2.5 μm 2.1×50 mm

Mobile phase: 5% Acetonitrile in Water (0.1% Formic acid) (A) and 95% Acetonitrile in Water (0.1% Formic acid) (B)

Gradient: flow rate at 0.6 mL/min

| Time (min) | A (%) | B (%) |
|---|---|---|
| 0.20 | 95.0 | 5.00 |
| 1.50 | 10.00 | 90.0 |
| 2.00 | 10.00 | 90.0 |
| 2.01 | 95.0 | 5.00 |
| 2.50 | 95.0 | 5.00 |

Injection volumn: 5 μL

### MS:

AB Sciex Triple Quad 5500 LC/MS/MS instrument (Serial NO. BB212611510)

### Quantification: Internal Standard Method

The plasma itraconazole concentrations and pharmacokinetic parameters after i.v. dosing of Compound 6 were summarized in Table 8 and 9.

TABLE 8
Plasma itroconazole concentrations after intravenous dosing
of Compound 6
IV Dose 0.822 mg/kg

| Time | Concentration (ng/mL) | | | Mean | SD | CV |
|---|---|---|---|---|---|---|
| (h) | Mouse 1 | Mouse 2 | Mouse 3 | (ng/mL) | (ng/mL) | (%) |
| 0.033 | 1020 | 735 | 907 | 887 | 144 | 16.2 |
| 0.25 | 105 | 107 | 104 | 105 | 2 | 1.45 |
| 0.5 | 78.9 | 80.5 | 64.4 | 74.6 | 8.9 | 11.9 |
| 1 | 75.6 | 83.2 | 77.9 | 78.9 | 3.9 | 4.94 |
| 2 | 43.0 | 44.3 | 29.5 | 38.9 | 8.2 | 21.0 |
| 4 | 10.6 | 18.9 | 9.83 | 13.1 | 5.0 | 38.4 |
| 6 | 4.08 | 8.95 | 5.99 | 6.34 | 2.45 | 38.7 |
| 8 | 1.92 | 4.20 | BLOQ | 3.06 | NA | NA |
| 24 | BLOQ | BLOQ | BLOQ | NA | NA | NA |

BLOQ: below limitation of quatification

TABLE 9
Itraconazole pharmacokinetic characteristics after intravenous
dosing of Compound 6

| PK parameters | Unit | Mouse 1 | Mouse 2 | Mouse 3 | Mean | SD | CV(%) |
|---|---|---|---|---|---|---|---|
| Cl_obs | mL/min/kg | 37.8 | 37.7 | 42.0 | 39.2 | 2.5 | 6.29 |
| T_{1/2} | h | 1.62 | 1.84 | 1.74 | 1.74 | 0.11 | 6.36 |
| C₀ | ng/mL | 1441 | 985 | 1261 | 1229 | 230 | 18.7 |
| AUCₗₐₛₜ | h*ng/mL | 358 | 352 | 311 | 340 | 26 | 7.52 |
| AUC_{Inf} | h*ng/mL | 362 | 363 | 326 | 351 | 21 | 6.07 |
| AUC_% Extrap_obs | % | 1.24 | 3.08 | 4.61 | 2.98 | 1.69 | 56.7 |
| MRT_{Inf}_obs | h | 1.12 | 1.74 | 1.26 | 1.37 | 0.32 | 23.5 |
| AUCₗₐₛₜ/D | h*mg/mL | 435 | 428 | 378 | 414 | 31 | 7.52 |
| Vₛₛ_obs | L/kg | 2.54 | 3.93 | 3.18 | 3.22 | 0.69 | 21.6 |

The plasma itraconazole concentrations and pharmacokinetic parameters after oral dosing of Compound 6 were summarized in Table 10 and 11.

TABLE 10
Plasma itroconazole concentrations after oral
dosing of Compound 6
PO Dose 4.11 mg/kg

| Time | Concentration (ng/mL) | | | Mean | SD | CV |
|---|---|---|---|---|---|---|
| (h) | Mouse 4 | Mouse 5 | Mouse 6 | (ng/mL) | (ng/mL) | (%) |
| 0.25 | 22.7 | 11.0 | 18.1 | 17.3 | 5.9 | 34.1 |
| 0.5 | 75.5 | 37.0 | 63.9 | 58.8 | 19.8 | 33.6 |
| 1 | 151 | 73.7 | 138 | 121 | 41 | 34.2 |
| 2 | 96.6 | 72.3 | 91.8 | 86.9 | 12.9 | 14.8 |
| 4 | 40.9 | 33.3 | 39.9 | 38.0 | 4.1 | 10.9 |
| 6 | 26.9 | 17.2 | 18.9 | 21.0 | 5.2 | 24.7 |
| 8 | 16.9 | 18.6 | 9.91 | 15.1 | 4.6 | 30.4 |
| 24 | BLOQ | BLOQ | BLOQ | NA | NA | NA |

BLOQ: below limitation of quatification

TABLE 11
Itraconazole pharmacokinetic characteristics after oral dosing of Compound 6

| PK parameters | Unit | Mouse 4 | Mouse 5 | Mouse 6 | Mean | SD | CV (%) |
|---|---|---|---|---|---|---|---|
| T_{1/2} | h | 3.14 | 2.93 | 1.99 | 2.69 | 0.61 | 22.7 |
| Tₘₐₓ | h | 1 | 1 | 1 | 1.00 | 0.00 | 0.00 |
| Cₘₐₓ | ng/mL | 151 | 73.7 | 138 | 121 | 41 | 34.2 |
| AUCₗₐₛₜ | h*ng/mL | 445 | 300 | 397 | 381 | 74 | 19.4 |
| AUC_{Inf} | h*ng/mL | 521 | 379 | 426 | 442 | 73 | 16.4 |
| AUC_% Extrap_obs | % | 14.7 | 20.8 | 6.69 | 14.0 | 7.1 | 50.3 |
| MRT_{Inf}_obs | h | 4.15 | 4.92 | 3.12 | 4.06 | 0.90 | 22.3 |
| AUCₗₐₛₜ/D | h*mg/mL | 108 | 73.0 | 96.7 | 93 | 18 | 19.4 |
| F | % | 29.8 | 17.6 | 24.3 | 23.9 | 6.1 | 25.4 |

The results of mouse PK study on Compound 6 showed that after intravenous dosing of 0.822 mpk, Itraconazole was released with an average AUC (area under the curve) of about 350 h*ng/mL, and with an average T_{1/2} of 1.7 hour.

The results of mouse PK study on Compound 6 showed that after oral dosing of 4.11 mpk, Itraconazole was released with an average AUC of about 400 h*ng/mL, and with an average T_{1/2} of 2.7 hour. Cmax was reached at about 1 hour after dosing and was at the level of about 120 ng/mL.

The data demonstrated that the compound of the invention when administered (intravenously, orally) delivered corresponding amount of Itraconazole in the blood.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An itraconazole prodrug compound represented by Formula (I), or a pharmaceutically acceptable salt or solvent thereof, 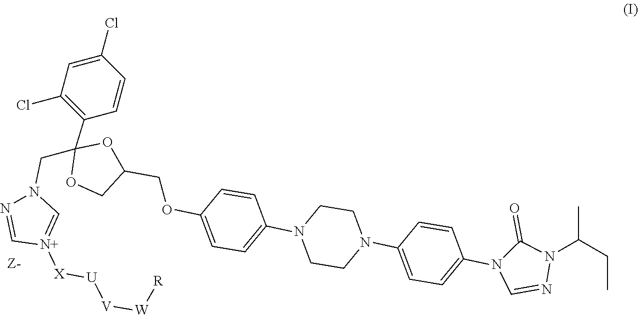
wherein X is omitted, or represents —CH₂— optionally substituted by C₁-C₅ alkyl,
U is omitted, or represents —O—, —S—, —NH—, or —N(R¹)—,
V is omitted, or represents —CO—, —SO—, —SO₂—, or 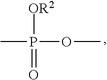
W is omitted, or represents —O—, —S—, —NH—, or —N(R³)—,
R represents H, C₁-C₅ alkyl, or hydroxyl-C₁-C₅ alkyl,
R¹, R² and R³ independently represents C₁-C₅ alkyl, or hydroxyl-C₁-C₅ alkyl, Z⁻ represents F⁻, Cl⁻, Br⁻, I⁻, PO₃⁻, SO₄⁻, HCO₂⁻, CH₃COO⁻, or CH₄H₂O₄²⁻.

2. The itraconazole prodrug compound of claim 1, wherein X is —CH₂—, U is —O—, V is —CO—, W is omitted, R is alkyl or hydroxyalkyl, and Z⁻ represents F⁻, Cl⁻, Br⁻, I⁻, PO₃⁻, SO₄⁻, HCO₂⁻, CH₃COO⁻, or CH₄H₂O₄²⁻.

3. The itraconazole prodrug compound of claim 2, wherein R is —CH₃, and Z⁻ is Cl⁻

4. The itraconazole prodrug compound of claim 2, wherein R is —CH(CH₃)CH₃, and Z⁻ is Cl⁻.

5. The itraconazole prodrug compound of claim 2, wherein R is —C(CH₃)₃, and Z⁻ is Cl⁻.

6. The itraconazole prodrug compound of claim 1, wherein X is —CH₂—, U is —O—, V is —CO—, W is —O—, R is alkyl or hydroxyalkyl, and Z⁻ represents F⁻, Cl⁻, Br⁻, I⁻, PO₃⁻, SO₄⁻, HCO₂⁻, CH₃COO⁻, or CH₄H₂O₄²⁻.

7. The itraconazole prodrug compound of claim 6, wherein R is —CH₂CH₃, and Z⁻ is Cl⁻.

8. The itraconazole prodrug compound of claim 6, wherein R is —CH₂CH₂CH₃, and Z⁻ is Cl⁻.

9. The itraconazole prodrug compound of claim 6, wherein R is —CH(CH₃)₂, and Z⁻ is Cl⁻.

10. The itraconazole prodrug compound of claim 6, wherein R is —CH₂CH₂CH₂OH, and Z⁻ is Cl⁻.

11. The itraconazole prodrug compound of claim 1, wherein X, U, V, and W are omitted, R is alkyl or hydroxyalkyl.

12. The itraconazole prodrug compound of claim 11, wherein R is —CH₃, and Z⁻ is Cl⁻.

13. The itraconazole prodrug compound of claim 11, wherein R is —CH₂CH₃, and Z⁻ is Cl⁻.

14. The itraconazole prodrug compound of claim 1, wherein X is —CH₂—, U is —O—, V is 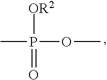
W is omitted, R and R² are independently alkyl or hydroxyalkyl, and Z⁻ represents F⁻, Cl⁻, Br⁻, I⁻, PO₃⁻, SO₄⁻, HCO₂⁻, CH₃COO⁻, or CH₄H₂O₄²⁻.

15. The itraconazole prodrug compound of claim 14, wherein R² is —CH₂CH₃, W is omitted, R is —CH₂CH₃, and Z⁻ is Cl⁻.

16. A pharmaceutical composition comprising a therapeutically effective amount of a compound of Formula (I), and a pharmaceutically acceptable carrier.

17. The pharmaceutical composition of claim 16, wherein the pharmaceutical composition is an inhaler dosage.

18. A method for treatment and/or prophylaxis of lung fibrosis, renal fibrosis, or liver fibrosis, comprising administering a subject in need thereof the pharmaceutical composition of claim 16.

19. The method of claim 19, wherein the lung fibrosis is idiopathic pulmonary fibrosis.

20. The itraconazole prodrug compound of claim 1, wherein the compound has a structure according to Formula A, 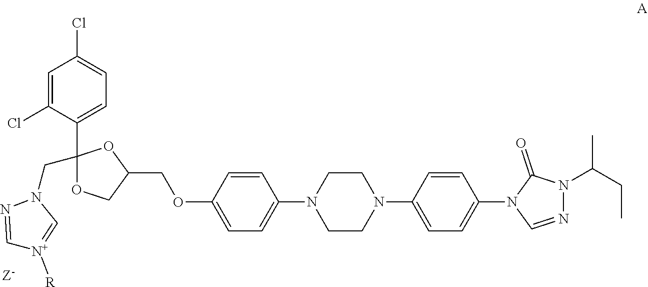
wherein X, U, V, and W are omitted, R is a C₁₋₅ alkyl and Z⁻ represents F⁻, Cl⁻, Br⁻, I⁻, or another pharmaceutically acceptable anion.

21. The itraconazole prodrug compound of claim 1, wherein the compound has a structure according to Formula B, 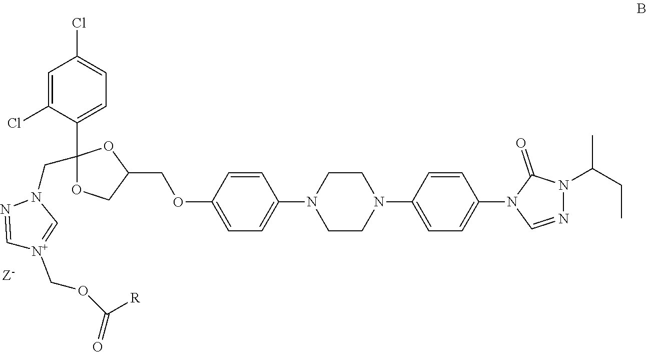
wherein X is —CH₂—, W is omitted and X, U, V, and R together form an ester, wherein R is a C₁₋₅ alkyl and Z⁻ represents F⁻, Cl⁻, Br⁻, I⁻, or another pharmaceutically acceptable anion.

22. The itraconazole prodrug compound of claim 1, wherein the compound has a structure according to Formula C, 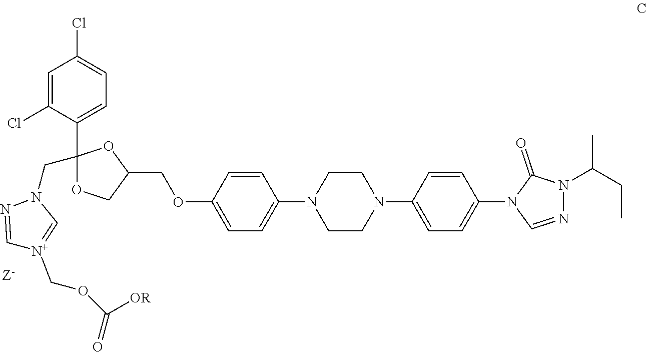
wherein X is —CH₂— and X, U, V, W, and R together form a carbonate ester, wherein R is a C₁₋₅ alkyl or hydroxyl-C₁-C₅ alkyl and Z⁻ represents F⁻, Cl⁻, Br⁻, I⁻, or another pharmaceutically acceptable anion.

23. The itraconazole prodrug compound of claim 1, wherein the compound has a structure according to Formula D, 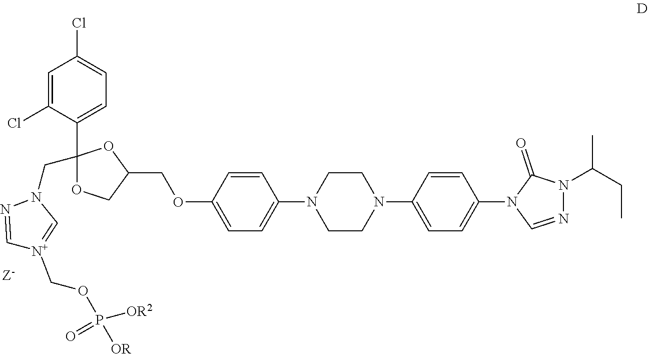
wherein X is —CH₂—, W is omitted, and U and V together form a phosphate, wherein R and R² are each independently a C₁₋₅ alkyl or C₁₋₅ hydroxyalkyl.
